(19) United States
(12) Patent Application Publication
(10) Pub. No.: US 2026/0367510 A1
(43) Pub. Date: Nov. 19, 2026

(54) RADIATION-SENSITIVE RESIN COMPOSITION AND PATTERN FORMATION METHOD

(71) Applicant: JSR CORPORATION, Tokyo (JP)

(72) Inventors: Motohiro SHIRATANI, Tokyo (JP); Asuka HACHIYA, Tokyo (JP)

(73) Assignee: JSR CORPORATION, Tokyo (JP)

(21) Appl. No.: 19/120,305

(22) PCT Filed: Sep. 5, 2023
(86) PCT No.: PCT/JP2023/032340

§ 371 Date: Apr. 11, 2025

(30) Foreign Application Priority Data
Oct. 24, 2022 (JP) 2022-169870

(51) Int. Cl. G03F 7/004; C07C 65/24; C07C 235/84; C07C 381/12; G03F 7/00; G03F 7/039; G03F 7/20; H10P 76/20

(52) U.S. Cl. CPC G03F 7/0045; C07C 65/24; C07C 235/84; C07C 381/12; G03F 7/0046; G03F 7/0397; H10P 76/2041; G03F 7/2002; G03F 7/70008

(57) ABSTRACT

A radiation-sensitive resin composition includes: one or more onium salts each including an organic acid anion moiety and an onium cation moiety; a resin including a structural unit including an acid-dissociable group; and a solvent. An onium cation moiety of at least a part of the one or more onium salts includes a fluorine atom. An organic acid anion moiety of at least a part of the one or more onium salts is represented by formula (1). R¹ is an organic group, and when q1 is two or more, two R¹s optionally taken together represent a ring structure having 5 to 20 ring numbers together with the carbon atoms of the benzene ring in the formula (1) to which the two R¹s are bonded. R² is a halogen atom, a hydroxy group, an alkyl group, an alkoxy group, a cyano group, a nitro group, or an amino group.

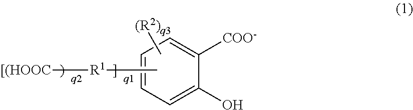

### TECHNICAL FIELD

The present invention relates to a radiation-sensitive resin composition and a pattern formation method.

### BACKGROUND ART

A photolithography technique performed using a resist composition has been utilized for formation of a fine circuit on a semiconductor device. As the representative procedure, for example, a resist pattern is formed on a substrate by generating an acid by irradiating the coating of the resist composition with radioactive ray through a mask pattern, and then generate a difference in solubility of resin into an alkaline or organic developer between an exposed part and a non-exposed part through a reaction in the presence of the acid as a catalyst.

In the photolithography technique, pattern miniaturization is promoted by using short-wavelength radiation such as ArF excimer laser, or by combining such radiation with an immersion exposure method (liquid immersion lithography). As the next generation technology, a shorter-wavelength radioactive-ray, such as an electron beam, an X-ray, and an extreme ultraviolet ray (EUV) is being utilized, and a resist material containing an acid generator with a fluorine-substituted benzene ring having an enhanced efficiency of absorbing such a radioactive-ray is also being studied (Patent Document 1).

### PRIOR ART DOCUMENT

### Patent Document

Patent Document 1: JP-A-2014-2359

### SUMMARY OF THE INVENTION

### Problems to be Solved by the Invention

However, it has been found that the storage stability of the resist composition is deteriorated when the technique for improving the absorption efficiency of radioactive-ray is applied.

An object of the present invention is to provide a radiation-sensitive resin composition having good storage stability and capable of forming a resist film having excellent sensitivity and line width roughness (LWR) performance when the next generation technology is applied, and a pattern formation method.

### Means for Solving the Problems

As a result of intensive research into solving the above problem, the present inventors discovered that the above object can be achieved by adopting the following configuration, and thus completed the present invention.

The present invention relates to, in one embodiment, a radiation-sensitive resin composition, including
one or two or more onium salts containing an organic acid anion moiety and an onium cation moiety;
a resin having a structural unit containing an acid-dissociable group; and
a solvent,
wherein at least a part of the onium cation moiety in the onium salt is a fluorine-containing onium cation moiety containing a fluorine atom, and
at least a part of the organic acid anion moiety in the onium salt is an organic acid anion moiety (1) represented by the following formula (1):

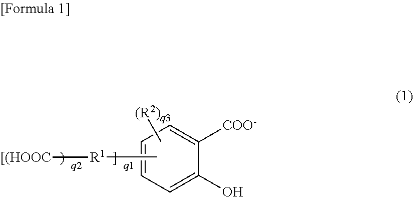

wherein in the formula (1),
R¹ is a (q2+1)-valent organic group, or represents, when there are a plurality of R¹s, a ring structure having 5 to 20 ring numbers formed together with a carbon atom of a benzene ring in the formula (1) to which two R¹s combined with each other are bonded,
R² is a halogen atom, a hydroxy group, an alkyl group, an alkoxy group, a cyano group, a nitro group, or an amino group,
q1 is an integer of 1 to 4, when q1 is 2 or more, a plurality of R¹s are identical to or different from each other,
when q1 is 1, q2 is an integer of 1 to 4, when q1 is 2 or more, a plurality of q2s are each independently an integer of 0 to 4, provided that at least one q2 is 1 or more,
q3 is an integer of 0 to 3, when q3 is 2 or more, a plurality of R²s are identical to or different from each other, and
q1 and q3 satisfy q1+q3≤4.

The radiation-sensitive resin composition has good storage stability, and can form a resist film satisfying sensitivity and LWR performance. The reason for this is not clear, but can be expected as follows. Due to the fluorine-containing onium cation moiety of the onium salt, the absorption of radioactive-ray such as EUV having a wavelength of 13.5 nm by fluorine atoms becomes very large, and the secondary electron generation efficiency also increases, so that the radiation-sensitive resin composition is made highly sensitive. In addition, since the onium salt has the organic acid anion moiety (1) having a salicylic acid derivative structure, the diffusion length of a generated acid and the acid capturing performance are appropriately controlled, and as a result, excellent patternability is exhibited. It is presumed that resist performances as described above can be exerted due to these combined actions.

On the other hand, in a system in which, for example, a salicylic acid anion that has been frequently used heretofore is simply applied as the organic acid anion moiety, the storage stability may be deteriorated by introduction of the fluorine-containing onium cation moiety. As a result of examining the cause, the present inventors have considered that the fluorine-containing onium cation has so high electrophilicity that storage stability is deteriorated when the fluorine-containing onium cation is used in combination with a highly basic organic acid anion. On the other hand, it is presumed that by adopting the organic acid anion moiety (1), decomposition of the fluorine-containing onium cation moiety can be suppressed to exhibit good storage stability.

The present invention relates to, in another embodiment, a pattern formation method including the steps of:
directly or indirectly applying the radiation-sensitive resin composition on a substrate to form a resist film;
exposing the resist film to light; and
developing the exposed resist film with a developer.

In the pattern formation method, since the radiation-sensitive resin composition capable of forming a resist film having good storage stability and excellent sensitivity and LWR performance is used, a high-quality resist pattern can be efficiently formed.

### MODE FOR CARRYING OUT THE INVENTION

Hereinafter, embodiments of the present invention will be described in detail, but the present invention is not limited to these embodiments.

### <<Radiation-Sensitive Resin Composition>>

The radiation-sensitive resin composition (hereinafter, also simply referred to as “composition”) according to the present embodiment contains one or two or more onium salts, a resin, and a solvent. The composition may contain another optional component as long as the effects of the present invention are not impaired. When the radiation-sensitive resin composition contains a predetermined onium salt, the radiation-sensitive resin composition can exhibit good storage stability, and can impart high level of sensitivity and LWR performance to an obtained resist film.

### <Onium Salt>

The onium salt is a component that contains an organic acid anion moiety and an onium cation moiety, and generates an acid through exposure to light. Since at least a part of the onium cation moiety in the onium salt is a fluorine-containing onium cation moiety containing a fluorine atom, high sensitivity can be achieved by improving the radioactive-ray absorption efficiency (and secondary electron generation efficiency) and then improving the acid generation efficiency.

The form of the onium salt contained in the radiation-sensitive resin composition is not particularly limited, but may be a form in which the onium salt structure exists alone as a (low molecular weight) compound, a form in which the onium salt structure is incorporated as a part of a polymer, or both of these forms. The radiation-sensitive resin composition preferably contains, as the onium salt, an acid diffusion controlling agent containing the organic acid anion moiety (1) and the onium cation moiety, and a radiation-sensitive acid generator containing an organic acid anion moiety (2) different from the organic acid anion moiety (1) and the onium cation moiety, which generates an acid having a pKa lower than that of an acid generated from the acid diffusion controlling agent by irradiation with radioactive-ray. The different functions of the acid diffusion controlling agent and the radiation-sensitive acid generator will be described below.

An acid generated through exposure of the onium salt to light is considered to have two functions in the radiation-sensitive resin composition depending on the strength of the acid. Examples of the first function include a function that, when a resin contains a structural unit having an acid-dissociable group, causes the acid generated through the exposure to dissociate the acid-dissociable group of the structural unit, to generate a carboxy group or the like. An onium salt having the first function is referred to as the radiation-sensitive acid generator. The second function includes a function that, under a pattern formation condition in which the radiation-sensitive resin composition is used, suppresses, by salt exchange, the diffusion of an acid generated from the radiation-sensitive acid generator in an unexposed area without substantially dissociating the acid-dissociable group of the resin. An onium salt having the second function is referred to as the acid diffusion controlling agent. The acid generated from the radiation-sensitive acid generator can be said to be a relatively stronger acid (acid having a lower pKa) than the acid generated from the acid diffusion controlling agent. Whether an onium salt functions as the radiation-sensitive acid generator or the acid diffusion controlling agent depends on the energy required for dissociating the acid-dissociable group of the resin, the acidity of the onium salt, and the like.

When the radiation-sensitive resin composition contains the radiation-sensitive acid generator, the polarity of the resin in an exposed area increases, and as a result, when the developer is an aqueous alkaline solution, the resin in the exposed area is soluble in the developer, and on the other hand, when the developer is an organic solvent, the resin in the exposed area is hardly soluble in the developer.

When the radiation-sensitive resin composition contains the acid diffusion controlling agent, diffusion of an acid in an unexposed area can be suppressed, and a resist pattern further superior in pattern developability and LWR performance can be formed.

In the present embodiment, at least one of the onium cation moiety in the acid diffusion controlling agent and the onium cation moiety in the radiation-sensitive acid generator is the fluorine-containing onium cation moiety. It is preferable that both the onium cation moiety in the acid diffusion controlling agent and the onium cation moiety in the radiation-sensitive acid generator be the fluorine-containing onium cation moiety.

The form of the fluorine atom contained in the fluorine-containing onium cation moiety is not particularly limited, but the fluorine-containing onium cation moiety preferably contains an aromatic ring structure having a fluorine atom (hereinafter, also referred to as a “fluorine-substituted aromatic ring structure”). By combining an electron-attracting fluorine atom with an aromatic ring structure, further improvement in radioactive-ray absorption efficiency can be expected. The “aromatic ring structure having a fluorine atom” includes not only a structure in which the fluorine atom is directly bonded to the aromatic ring structure but also a structure in which the fluorine atom is bonded to the aromatic ring structure via another atom (for example, a structure in which the fluorine atom is bonded to a substituent bonded to the aromatic ring structure).

### <Acid Diffusion Controlling Agent>

The acid diffusion controlling agent contains the organic acid anion moiety (1) represented by the formula (1) and the onium cation moiety. The onium cation moiety may be a fluorine-containing onium cation moiety or an onium cation moiety having no fluorine atom, but is preferably a fluorine-containing onium cation moiety. A part of the acid diffusion controlling agent may contain the fluorine-containing onium cation part, and the remainder may contain the onium cation part having no fluorine atom.

The (q2+1)-valent organic group represented by R¹ is not particularly limited, and examples thereof include a group obtained by removing (q2−1) hydrogen atoms from a monovalent organic group having 1 to 40 carbon atoms. The monovalent organic group having 1 to 40 carbon atoms may be a group containing any of a chain structure, a cyclic structure, or a combination thereof. Examples of the chain structure include a chain hydrocarbon group that may either be saturated or unsaturated and linear or branched. Examples of the cyclic structure include a cyclic hydrocarbon group that may be alicyclic, aromatic, or heterocyclic. Among them, the monovalent organic group is preferably a monovalent chain hydrocarbon group having 1 to 20 carbon atoms, a monovalent alicyclic hydrocarbon group having 3 to 20 carbon atoms, a monovalent aromatic hydrocarbon group having 6 to 20 carbon atoms, or a combination thereof. In particular, the monovalent organic group is preferably a group containing a monovalent aromatic hydrocarbon group having 6 to 20 carbon atoms. Further examples include a group obtained by substituting, with a substituent, some or all of the hydrogen atoms contained in a group having a chain structure or a group having a cyclic structure, and such a group but further containing, between carbon atoms or at a carbon-chain end, —CO—, —CS—, —O—, —S—, —SO₂—, —NR′—, or a combination of two or more thereof (R′ is a hydrogen atom or a monovalent hydrocarbon group having 1 to 10 carbon atoms).

Examples of the substituent that substitutes some or all of hydrogen atoms in the organic group include halogen atoms such as a fluorine atom, a chlorine atom, a bromine atom, and an iodine atom; a hydroxy group; a carboxy group; a cyano group; a nitro group; an alkyl group, an alkoxy group, an alkoxycarbonyl group, an alkoxycarbonyloxy group, an acyl group, an acyloxy group, or groups obtained by substituting a hydrogen atom of these groups with a halogen atom; and an oxo group (═O).

Examples of the monovalent chain hydrocarbon group having 1 to 20 carbon atoms may include a linear or branched saturated hydrocarbon group having 1 to 20 carbon atoms, or a linear or branched unsaturated hydrocarbon group having 1 to 20 carbon atoms.

Examples of the monovalent alicyclic hydrocarbon group having 3 to 20 carbon atoms include a monocyclic or polycyclic saturated hydrocarbon group, or a monocyclic or polycyclic unsaturated hydrocarbon group. As the monocyclic saturated hydrocarbon group, a cyclopentyl group, a cyclohexyl group, a cycloheptyl group, and a cyclooctyl group are preferable. As the polycyclic cycloalkyl group, bridged alicyclic hydrocarbon groups such as a norbornyl group, an adamantyl group, a tricyclodecyl group, and a tetracyclododecyl group are preferable. The bridged alicyclic hydrocarbon group refers to a polycyclic alicyclic hydrocarbon group in which two carbon atoms among carbon atoms composing an alicyclic ring that are not adjacent to each other are bonded by a bonding chain containing one or more carbon atoms.

Examples of the monovalent aromatic hydrocarbon group having 6 to 20 carbon atoms include: aryl groups such as a phenyl group, a tolyl group, a xylyl group, a naphthyl group, and an anthryl group; and aralkyl groups such as a benzyl group, a phenethyl group, and a naphthylmethyl group.

Examples of the heterocyclic cyclic hydrocarbon group include a group obtained by removing one hydrogen atom from an aromatic heterocyclic structure and a group obtained by removing one hydrogen atom from an aliphatic heterocyclic structure. A 5-membered aromatic structure having aromaticity due to introduction of a heteroatom is also included in the heterocyclic structure. Examples of the hetero atom include an oxygen atom, a nitrogen atom, and a sulfur atom. The heterocyclic cyclic hydrocarbon group also includes a lactone structure, a cyclic carbonate structure, and a sultone structure.

Examples of the aromatic heterocyclic structure include:
oxygen atom-containing aromatic heterocyclic structures such as furan, pyran, benzofuran, and benzopyran;
nitrogen atom-containing aromatic heterocyclic structures such as pyrrole, imidazole, pyridine, pyrimidine, pyrazine, indole, quinoline, isoquinoline, acridine, phenazine, and carbazole;
sulfur atom-containing aromatic heterocyclic structures such as thiophene; and
aromatic heterocyclic structures containing a plurality of heteroatoms such as thiazole, benzothiazole, thiazine, and oxazine.

Examples of the aliphatic heterocyclic structure include:
oxygen atom-containing aliphatic heterocyclic structures such as oxirane, tetrahydrofuran, tetrahydropyran, dioxolane, and dioxane;
nitrogen atom-containing aliphatic heterocyclic structures such as aziridine, pyrrolidine, piperidine and piperazine;
sulfur atom-containing aliphatic heterocyclic structures such as thietane, thiolane, and thiane; and
aliphatic heterocyclic structures containing a plurality of heteroatoms such as morpholine, 1,2-oxathiolane, and 1,3-oxathiolane.

Examples of the lactone structure, cyclic carbonate structure, and sultone structure include structures represented by the following formulas (H-1) to (H-10).

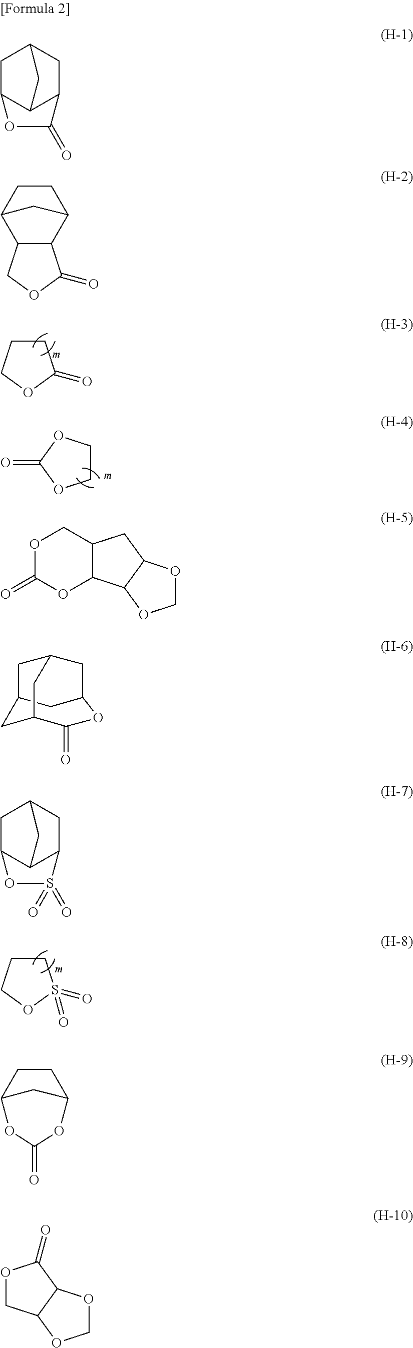

In the formulas, m is an integer of 1 to 3.

The ring structure having 5 to 20 ring numbers formed together with a carbon atom of the benzene ring in the formula (1) to which two R¹s combined with each other are bonded forms a fused ring together with the benzene ring. In the present description, the “fused ring” is a ring structure in which adjacent rings share one side (two adjacent atoms). Examples of the fused ring include aromatic hydrocarbon rings such as an indene ring (a fused ring of cyclopentadiene and benzene, and hereinafter, a fused ring of a benzene ring and another (aromatic) ring in the same manner), a naphthalene ring, an anthracene ring, and a fluorene ring, aromatic heterocyclic rings such as a quinoline ring, an isoquinoline ring, a quinoxaline ring, a quinazoline ring, a cinnoline ring, a benzofuran ring, an isobenzofuran ring, an indole ring, an isoindole ring, a benzothiophene ring, a benzimidazole ring, an indazole ring, a benzoxazole ring, a benzisoxazole ring, and a benzothiazole ring, or a combination thereof. Among them, a benzene ring (a naphthalene ring as a fused ring) is preferable as the ring structure having 5 to 20 ring members.

In the formula (1), R¹ preferably has an aromatic ring structure. Examples of the aromatic ring structure include a structure corresponding to the monovalent aromatic hydrocarbon group having 6 to 20 carbon atoms, a structure corresponding to the heterocyclic cyclic hydrocarbon group, or a combination thereof. As the aromatic ring structure, a benzene ring is preferable.

When R¹ includes an aromatic ring structure, the aromatic ring structure preferably has an iodine atom. Since the absorption of radioactive-ray such as EUV having a wavelength of 13.5 nm by an iodine atom is very large, the sensitivity of the resist film obtained from the radiation-sensitive resin composition can be enhanced.

Examples of the halogen atom represented by R² include a fluorine atom, a chlorine atom, an iodine atom, and a bromine atom. Examples of the alkyl group represented by R² include linear or branched alkyl groups having 1 to 10 carbon atoms such as a methyl group, an ethyl group, an n-propyl group, an i-propyl group, an n-butyl group, an i-butyl group, and a t-butyl group. Examples of the alkoxy group represented by R² include linear or branched alkoxy groups having 1 to 10 carbon atoms such as a methoxy group and an ethoxy group.

R² is preferably a halogen atom or an alkoxy group, more preferably an iodine atom or a methoxy group.

q1 is preferably an integer of 1 to 3, more preferably 1 or 2.

When q1 is 1, q2 is preferably an integer of 1 to 3, more preferably 1 or 2. When q1 is 2 or more, a plurality of q2s are each independently preferably an integer of 0 to 3, more preferably an integer of 0 to 2, still more preferably 0 or 1. At least one q2 of the plurality of q2s is preferably 1.

q3 is preferably an integer of 0 to 2, more preferably 0 or 1.

Examples of the organic acid anion moiety of the acid diffusion controlling agent represented by the formula (1) include, but are not limited to, structures represented by the following formulas (1-1) to (1-21).

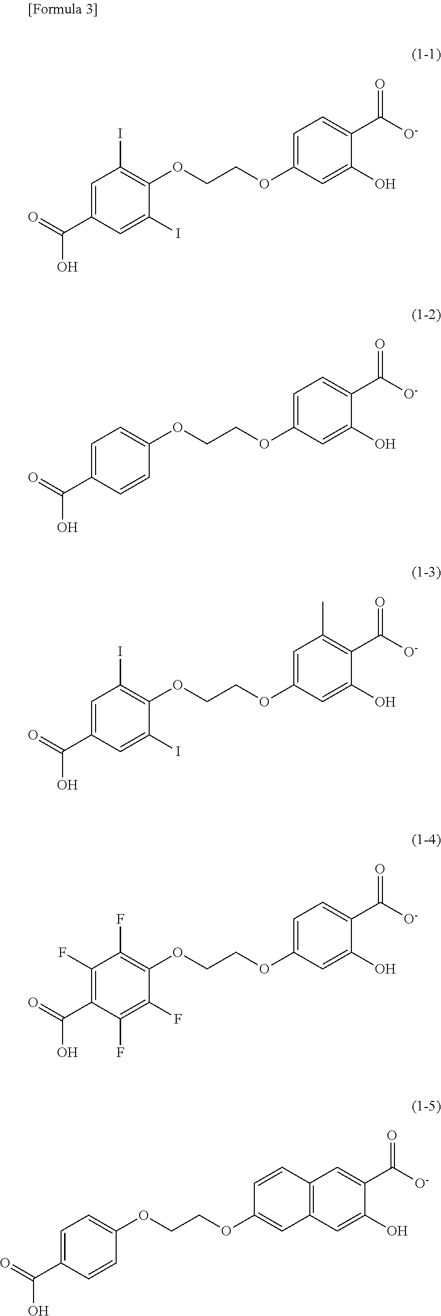 [SMILES: O=C(O)c1cc(I)c(OCCOc2ccc(C(=O)[O-])c(O)c2)c(I)c1 ; O=C(O)c1ccc(OCCOc2ccc(C(=O)[O-])c(O)c2)cc1 ; Cc1cc(OCCOc2c(I)cc(C(=O)O)cc2I)cc(O)c1C(=O)[O-] ; O=C([O-])c1ccc(OCCOc2c(F)c(F)c(C(=O)O)c(F)c2F)cc1O ; O=C(O)c1ccc(OCCOc2ccc3cc(C(=O)[O-])c(O)cc3c2)cc1]

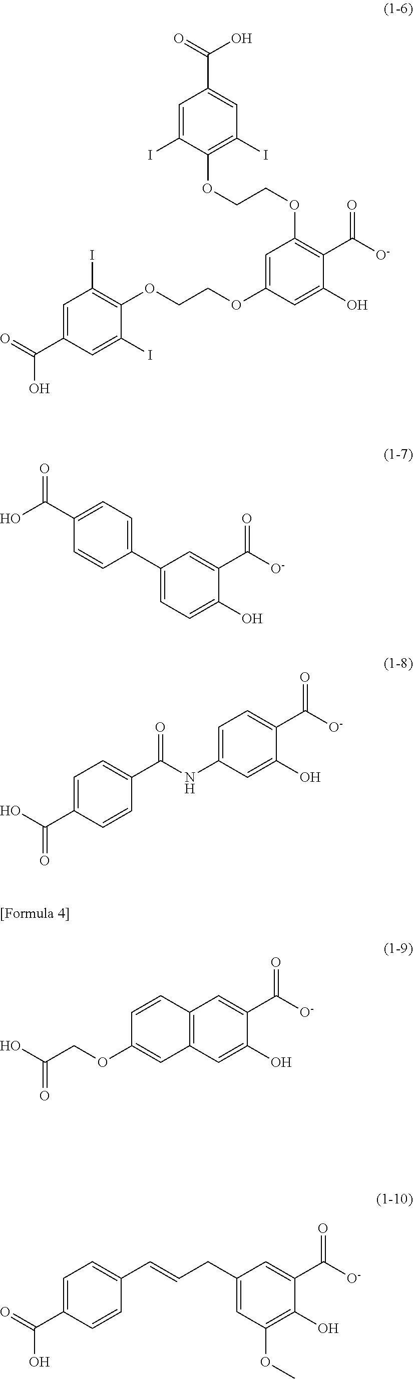 [SMILES: O=C(O)c1cc(I)c(OCCOc2cc(O)c(C(=O)[O-])c(OCCOc3c(I)cc(C(=O)O)cc3I)c2)c(I)c1 ; O=C(O)c1ccc(-c2ccc(O)c(C(=O)[O-])c2)cc1 ; O=C(O)c1ccc(C(=O)Nc2ccc(C(=O)[O-])c(O)c2)cc1 ; O=C(O)COc1ccc2cc(C(=O)[O-])c(O)cc2c1 ; COc1cc(C/C=C/c2ccc(C(=O)O)cc2)cc(C(=O)[O-])c1O]

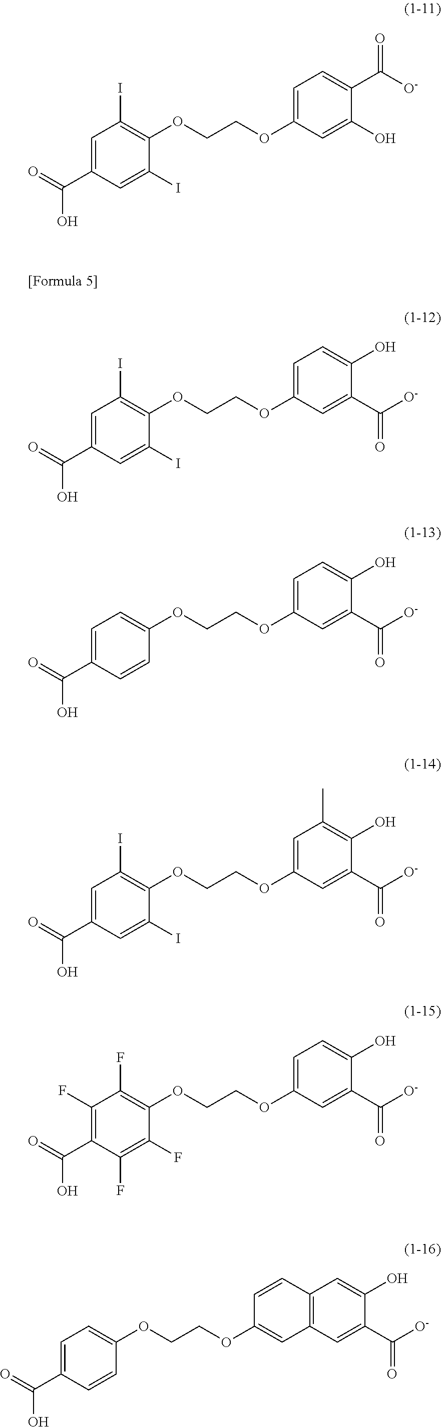 [SMILES: O=C(O)c1cc(I)c(OCCOc2ccc(C(=O)[O-])c(O)c2)c(I)c1 ; O=C(O)c1cc(I)c(OCCOc2ccc(O)c(C(=O)[O-])c2)c(I)c1 ; O=C(O)c1ccc(OCCOc2ccc(O)c(C(=O)[O-])c2)cc1 ; Cc1cc(OCCOc2c(I)cc(C(=O)O)cc2I)cc(C(=O)[O-])c1O ; O=C([O-])c1cc(OCCOc2c(F)c(F)c(C(=O)O)c(F)c2F)ccc1O ; O=C(O)c1ccc(OCCOc2ccc3cc(O)c(C(=O)[O-])cc3c2)cc1]

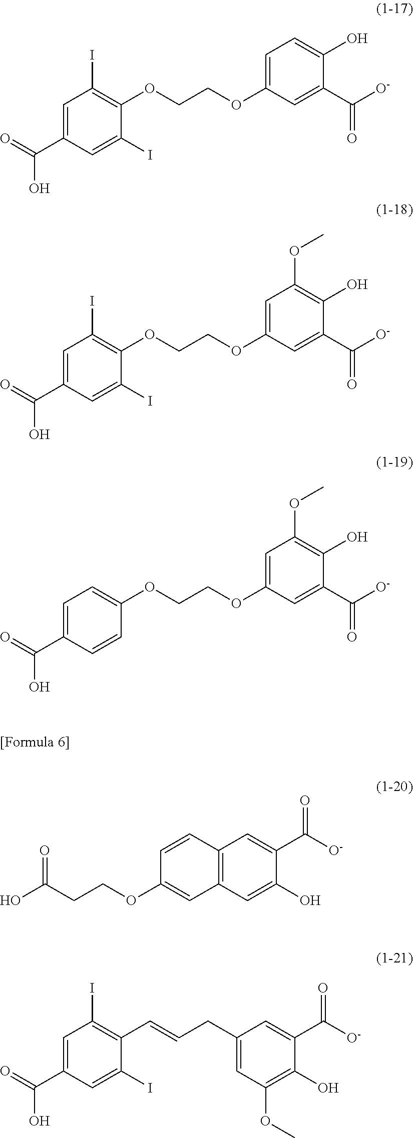 [SMILES: O=C(O)c1cc(I)c(OCCOc2ccc(O)c(C(=O)[O-])c2)c(I)c1 ; COc1cc(OCCOc2c(I)cc(C(=O)O)cc2I)cc(C(=O)[O-])c1O ; COc1cc(OCCOc2ccc(C(=O)O)cc2)cc(C(=O)[O-])c1O ; O=C(O)CCOc1ccc2cc(C(=O)[O-])c(O)cc2c1 ; COc1cc(C/C=C/c2c(I)cc(C(=O)O)cc2I)cc(C(=O)[O-])c1O]

The onium cation moiety of the acid diffusion controlling agent is preferably represented by the following formula (Q-1).

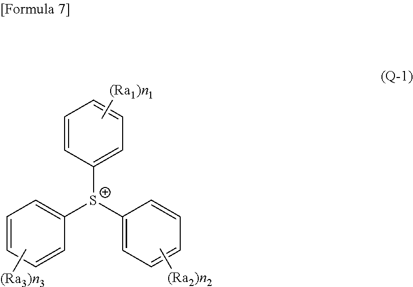

In the formula (Q-1), Ra1 and Ra2 each independently represent a substituent. n1 represents an integer of 0 to 5, and when n1 is 2 or more, the plurality of Ra1s may be the same as or different from each other. n2 represents an integer of 0 to 5, and when n2 is 2 or more, the plurality of Ra2s may be the same as or different from each other. n3 represents an integer of 0 to 5, and when n3 is 2 or more, the plurality of Ra3s may be the same as or different from each other. Ra3 represents a fluorine atom or a group having one or more fluorine atoms. Ra1 and Ra2 may be linked to each other to form a ring. When n1 is 2 or more, the plurality of Ra1s may be linked to each other to form a ring. When n2 is 2 or more, the plurality of Ra2s may be linked to each other to form a ring.

The substituent represented by Ra1, Ra2 and Ra3 is preferably an alkyl group, a cycloalkyl group, an alkoxy group, a cycloalkyloxy group, an alkoxycarbonyl group, an alkylsulfonyl group, a hydroxy group, a halogen atom, or a halogenated hydrocarbon group.

The alkyl group as Ra1 and Ra2 may be either a linear alkyl group or a branched alkyl group. The alkyl group is preferably one having 1 to 10 carbon atoms, and examples thereof include a methyl group, an ethyl group, an n-propyl group, an i-propyl group, an n-butyl group, a 2-methylpropyl group, a 1-methylpropyl group, a t-butyl group, an n-pentyl group, a neopentyl group, an n-hexyl group, an n-heptyl group, an n-octyl group, a 2-ethylhexyl group, an n-nonyl group, and an n-decyl group. Among them, a methyl group, an ethyl group, an n-butyl group, and a t-butyl group are particularly preferable.

Examples of the cycloalkyl group as Ra1 and Ra2 include monocyclic or polycyclic cycloalkyl groups (preferably cycloalkyl groups having 3 to 20 carbon atoms), and examples thereof include a cyclopropyl group, a cyclobutyl group, a cyclopentyl group, a cyclohexyl group, a cycloheptyl group, a cyclooctyl group, a cyclododecanyl group, a cyclopentenyl group, a cyclohexenyl group, and a cyclooctadienyl group. Among them, a cyclopropyl group, a cyclopentyl group, a cyclohexyl group, a cyclohexyl group, a cycloheptyl group, and a cyclooctyl group are particularly preferable.

Examples of the alkyl group moiety of the alkoxy group as Ra1 and Ra2 include those listed above as the alkyl group as Ra1 and Ra2. As the alkoxy group, a methoxy group, an ethoxy group, an n-propoxy group, and an n-butoxy group are particularly preferable.

Examples of the cycloalkyl group moiety of the cycloalkyloxy group as Ra1 and Ra2 include those listed above as the cycloalkyl group as Ra1 and Ra2. As the cycloalkyloxy group, a cyclopentyloxy group and a cyclohexyloxy group are particularly preferable.

Examples of the alkoxy group moiety of the alkoxycarbonyl group as Ra1 and Ra2 include those listed above as the alkoxy group as Ra1 and Ra2. As the alkoxycarbonyl group, a methoxycarbonyl group, an ethoxycarbonyl group, and an n-butoxycarbonyl group are particularly preferable.

Examples of the alkyl group moiety of the alkylsulfonyl group as Ra1 and Ra2 include those listed above as the alkyl group as Ra1 and Ra2. Examples of the cycloalkyl group moiety of the cycloalkylsulfonyl group as Ra1 and Ra2 include those listed above as the cycloalkyl group as Ra1 and Ra2. As the alkylsulfonyl group or the cycloalkylsulfonyl group, a methanesulfonyl group, an ethanesulfonyl group, an n-propanesulfonyl group, an n-butanesulfonyl group, a cyclopentanesulfonyl group, and a cyclohexanesulfonyl group are particularly preferable.

Each of the groups Ra1 and Ra2 may further have a substituent. Examples of the substituent include a halogen atom such as a fluorine atom (preferably a fluorine atom), a hydroxy group, a carboxy group, a cyano group, a nitro group, an alkoxy group, a cycloalkyloxy group, an alkoxyalkyl group, a cycloalkyloxyalkyl group, an alkoxycarbonyl group, a cycloalkyloxycarbonyl group, an alkoxycarbonyloxy group, and a cycloalkyloxycarbonyloxy group.

Examples of the halogen atom as Ra1 and Ra2 include a fluorine atom, a chlorine atom, a bromine atom, and an iodine atom, and a fluorine atom is preferable.

As the halogenated hydrocarbon group as Ra1 and Ra2, a halogenated alkyl group is preferable. Examples of the alkyl group and the halogen atom composing the halogenated alkyl group include those described above. Among them, a fluorinated alkyl group is preferable, and CF₃ is more preferable.

As described above, Ra₁ and Ra₂ may be linked to each other to form a ring (that is, a heterocyclic ring containing a sulfur atom). In this case, Ra₁ and Ra₂ preferably form a single bond or a divalent linking group, and examples of the divalent linking group include —COO—, —OCO—, —CO—, —O—, —S—, —SO—, —SO₂—, an alkylene group, a cycloalkylene group, an alkenylene group, or a combination of two or more thereof, and those having a total carbon number of 20 or less are preferable. When Ra¹ and Ra² are linked to each other to form a ring, Ra¹ and Ra² preferably form —COO—, —OCO—, —CO—, —O—, —S—, —SO—SO₂—, or a single bond, more preferably form —O—, —S—, or a single bond, particularly preferably form a single bond. When n1 is 2 or more, a plurality of Ra₁s may be linked to each other to form a ring, and when n2 is 2 or more, a plurality of Ra₂s may be linked to each other to form a ring. Examples thereof include an aspect in which two Ra₁s are linked to each other to form a naphthalene ring together with a benzene ring to which two Ra₁s are bonded.

Ra3 is preferably a fluorine atom or a group having a fluorine atom. Examples of the group having a fluorine atom may include groups in which a halogen atom of the alkyl group, cycloalkyl group, alkoxy group, cycloalkyloxy group, alkoxycarbonyl group, and alkylsulfonyl group as Ra1 and Ra2 is substituted with a fluorine atom. Among them, fluorinated alkyl groups are suitable, CF₃, C₂F₅, C₃F₇, C₄F₉, C₅F₁₁, C₆F₁₃, C₇F₁₅, C₈F₁₇, CH₂CF₃, CH₂CH₂CF₃, CH₂C₂F₅, CH₂CH₂C₂F₅, CH₂C₃F₇, CH₂CH₂C₃F₇, CH₂C₄F₉, and CH₂CH₂C₄F₉ are more suitable, and CF₃ is particularly suitable.

Ra3 is preferably a fluorine atom, or CF₃, more preferably a fluorine atom.

n1 and n2 are each independently preferably an integer of 0 to 3, preferably an integer of 0 to 2.

n3 is preferably an integer of 1 to 3, more preferably 1 or 2.

(n1+n2+n3) is preferably an integer of 1 to 15, more preferably an integer of 1 to 9, still more preferably an integer of 2 to 6, particularly preferably an integer of 3 to 6. When (n1+n2+n3) is 1, it is preferable that n3=1, and Ra3 be a fluorine atom, or CF₃. When (n1+n2+n3) is 2, a combination in which n1=n3=1, and Ra1 and Ra3 are each independently a fluorine atom, or CF₃, and a combination in which n3=2, and Ra3 is a fluorine atom, or CF₃ are preferable. When (n1+n2+n3) is 3, a combination in which n1=n2=n3=1, and Ra1 to Ra3 are each independently a fluorine atom, or CF₃ is preferable. When (n1+n2+n3) is 4, a combination in which n1=n3=2, and Ra1 and Ra3 are each independently a fluorine atom, or CF₃ is preferable. When (n1+n2+n3) is 5, a combination in which n1=n2=1 and n3=3, and Ra1 to Ra3 are each independently a fluorine atom, or CF₃, a combination in which n1=n2=2 and n3=1, and Ra1 to Ra3 are each independently a fluorine atom, or CF₃, and a combination in which n3=5, and Ra3s are each independently a fluorine atom, or CF₃ are preferable. When (n1+n2+n3) is 6, a combination in which n1=n2=n3=2, and Ra1 to Ra3 are each independently a fluorine atom, or CF₃ is preferable.

Specific examples of such an onium cation moiety represented by the formula (Q-1) include the following. While most of those shown below are sulfonium cation moieties having a fluorine-substituted aromatic ring structure, onium cation moieties having no fluorine-substituted aromatic ring structure that can be suitably employed include structures in which the fluorine atoms or CF₃ in the formulas shown below are substituted with an atom or a group other than a fluorine atom such as a hydrogen atom or another substituent.

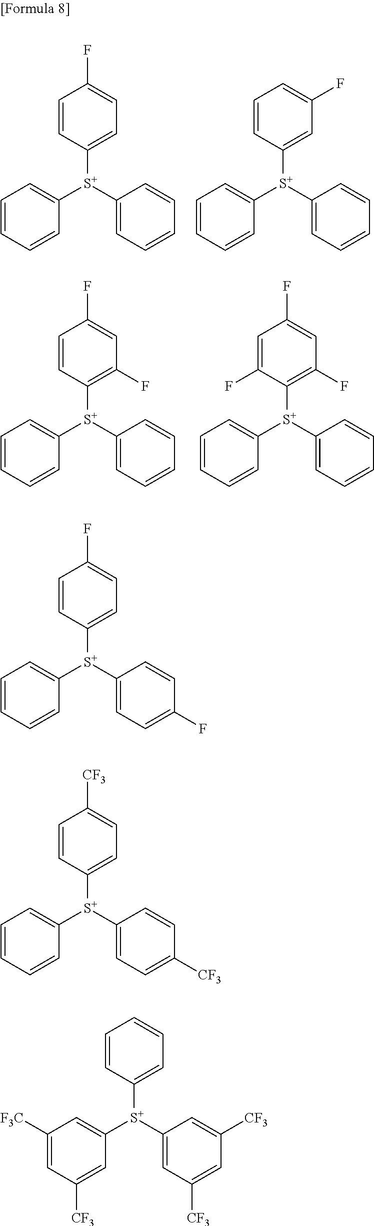

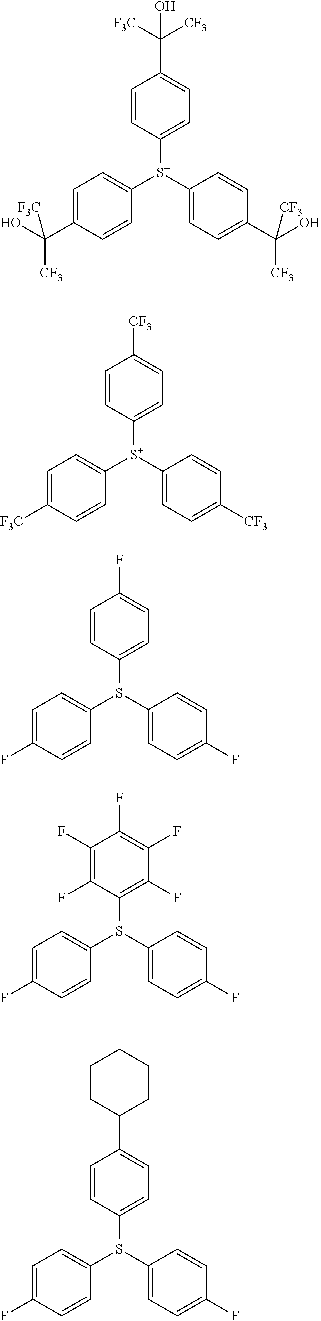 [SMILES: OC(c1ccc([S+](c2ccc(C(O)(C(F)(F)F)C(F)(F)F)cc2)c2ccc(C(O)(C(F)(F)F)C(F)(F)F)cc2)cc1)(C(F)(F)F)C(F)(F)F ; FC(F)(F)c1ccc([S+](c2ccc(C(F)(F)F)cc2)c2ccc(C(F)(F)F)cc2)cc1 ; Fc1ccc([S+](c2ccc(F)cc2)c2ccc(F)cc2)cc1 ; Fc1ccc([S+](c2ccc(F)cc2)c2c(F)c(F)c(F)c(F)c2F)cc1 ; Fc1ccc([S+](c2ccc(F)cc2)c2ccc(C3CCCCC3)cc2)cc1]

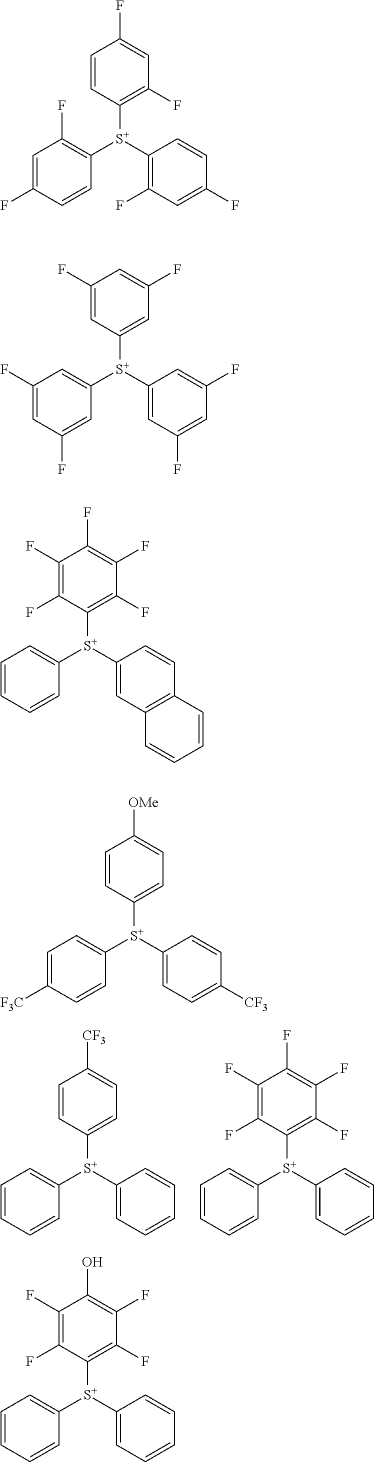

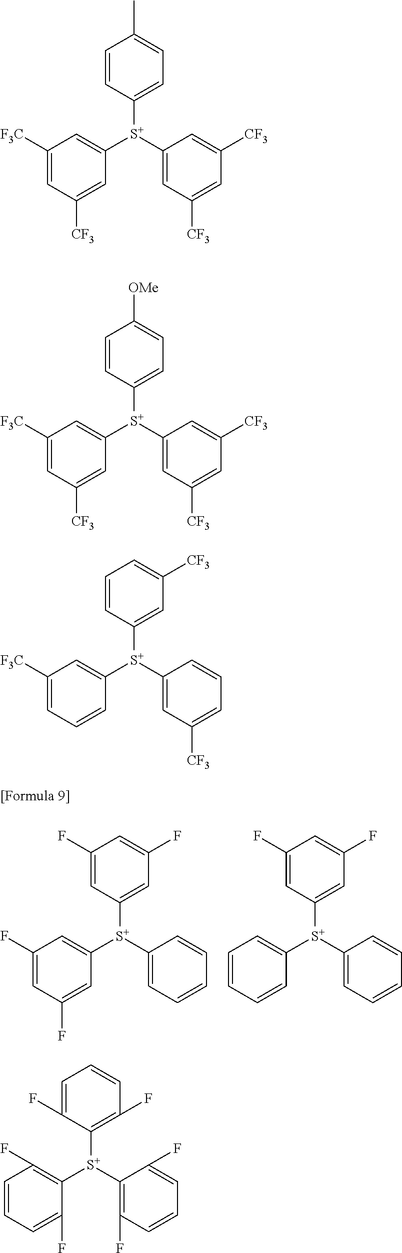 [SMILES: Cc1ccc([S+](c2cc(C(F)(F)F)cc(C(F)(F)F)c2)c2cc(C(F)(F)F)cc(C(F)(F)F)c2)cc1 ; COc1ccc([S+](c2cc(C(F)(F)F)cc(C(F)(F)F)c2)c2cc(C(F)(F)F)cc(C(F)(F)F)c2)cc1 ; FC(F)(F)c1cccc([S+](c2cccc(C(F)(F)F)c2)c2cccc(C(F)(F)F)c2)c1 ; Fc1cc(F)cc([S+](c2ccccc2)c2cc(F)cc(F)c2)c1 ; Fc1cc(F)cc([S+](c2ccccc2)c2ccccc2)c1 ; Fc1cccc(F)c1[S+](c1c(F)cccc1F)c1c(F)cccc1F]

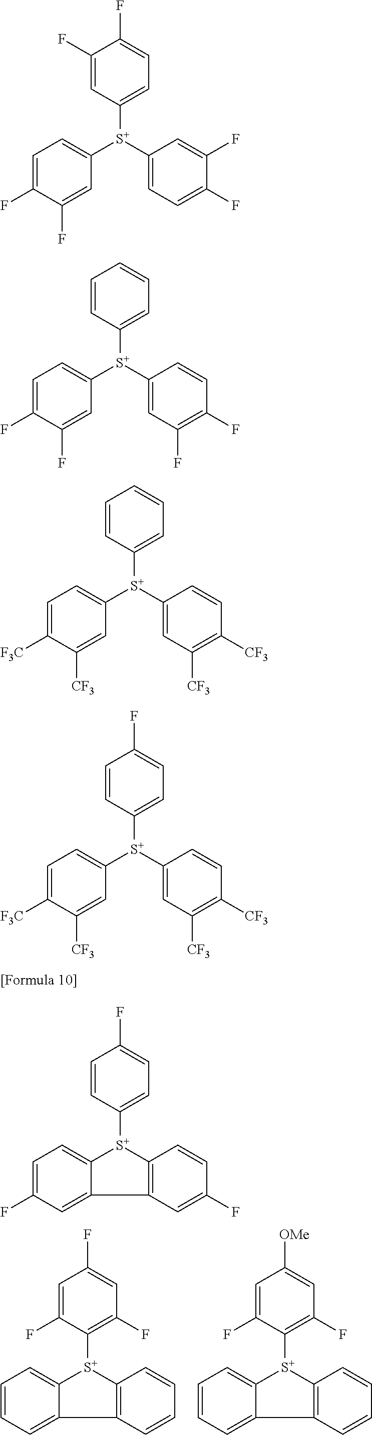

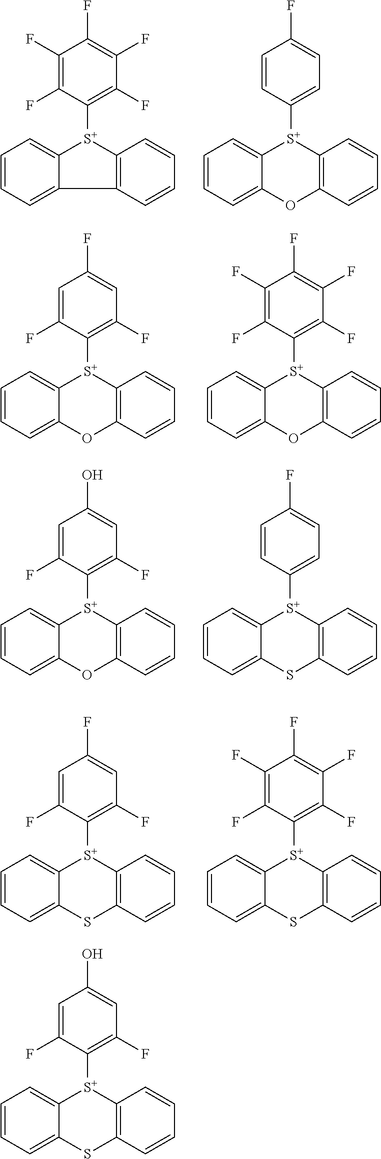

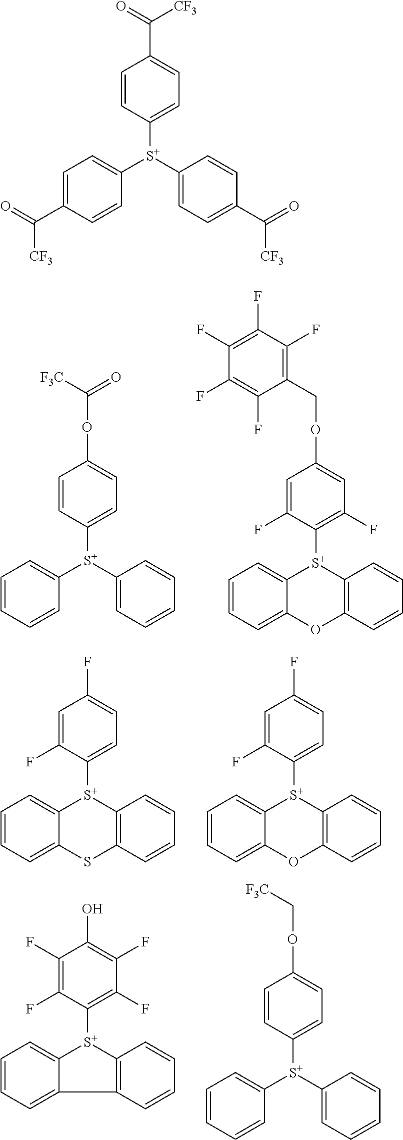

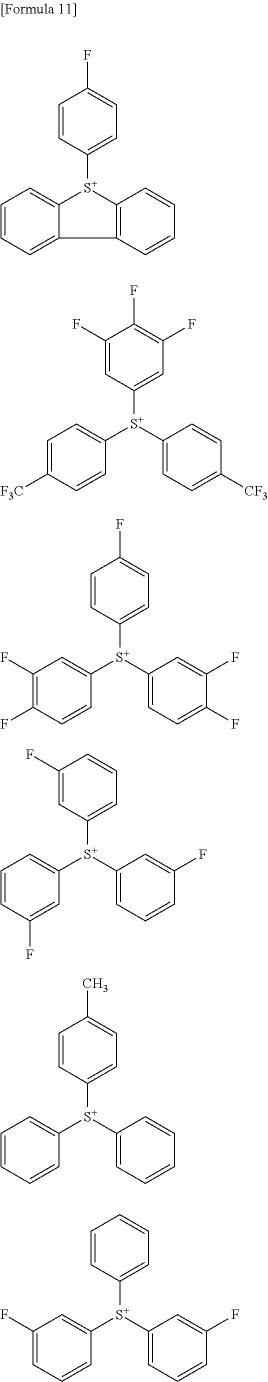 [SMILES: Fc1ccc(-[s+]2c3ccccc3c3ccccc32)cc1 ; Fc1cc([S+](c2ccc(C(F)(F)F)cc2)c2ccc(C(F)(F)F)cc2)cc(F)c1F ; Fc1ccc([S+](c2ccc(F)c(F)c2)c2ccc(F)c(F)c2)cc1 ; Fc1cccc([S+](c2cccc(F)c2)c2cccc(F)c2)c1 ; Cc1ccc([S+](c2ccccc2)c2ccccc2)cc1 ; Fc1cccc([S+](c2ccccc2)c2cccc(F)c2)c1]

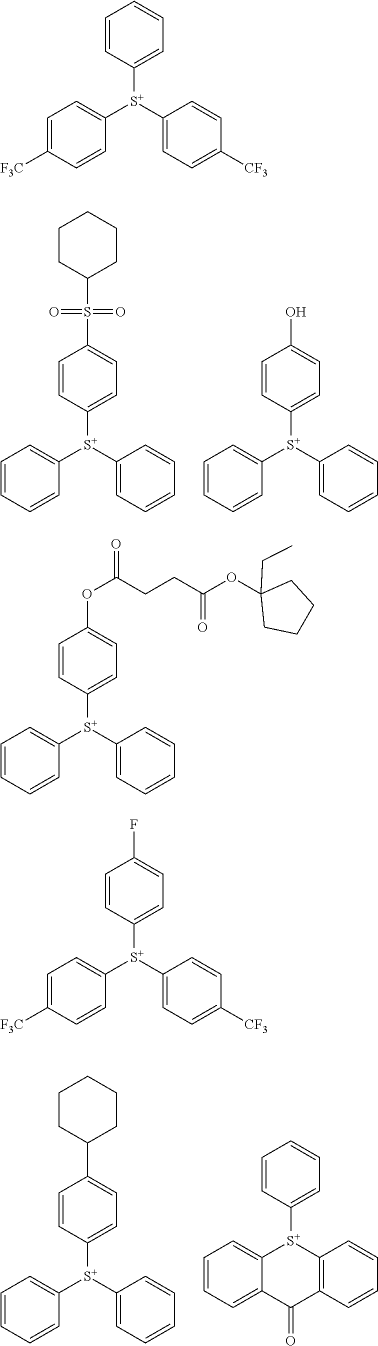

When the onium cation moiety of the acid diffusion controlling agent contains a fluorine-substituted aromatic ring structure, the onium cation moiety may be a diaryliodonium cation having one or more fluorine atoms.

Examples of the onium salt as the acid diffusion controlling agent include a structure in which the organic acid anion moiety (1) and the onium cation moiety are arbitrarily combined. When the onium salt as an acid diffusion controlling agent is incorporated as a part of a polymer (that is, a resin), a monomer in which a polymerizable group such as a vinyl group or a (meth)acryloyl group is introduced into the organic acid anion moiety (1) or the onium cation moiety is prepared, and the monomer can be incorporated into a resin by polymerization.

The acid diffusion controlling agent can be synthesized by a known method. Examples thereof include a method in which an organic acid anion moiety (1) having the hydrocarbon as a linking group is produced by a sequential nucleophilic substitution reaction of a dihalogenated hydrocarbon with hydroxysalicylic acid and hydroxybenzoic acid, and a salt exchange reaction is performed at the end. Examples of other synthesis methods include a method in which an organic acid anion moiety (1) is generated by a cross-coupling reaction between carboxyphenylboronic acid and halogenated salicylic acid, and a salt exchange reaction is performed at the end, and a method in which an organic acid anion moiety (1) is generated by an amidation reaction between an acid halide of benzoic acid and aminosalicylic acid, and a salt exchange reaction is performed at the end. In any method, a carboxy group may be obtained by protecting a carboxy group as an ester form or the like, then allowing each reaction to proceed, and deprotecting the carboxy group at a later stage.

A known acid diffusion controlling agent may also be used in combination as long as the effect of the present invention is not impaired. In addition, a case where the organic acid anion moiety and the onium cation moiety share the same aromatic ring structure is also included in the acid diffusion controlling agent of the present embodiment.

The lower limit of the content of the onium salt (in the case of a combination of two or more types, the total content of them) is preferably 5 mol %, more preferably 10 mol %, still more preferably 15 mol % with respect to the number of moles of the radiation-sensitive acid generator described later. The upper limit of the content is preferably 40 mol %, more preferably 30 mol %, still more preferably 25 mol %. The content of the onium salt is appropriately chosen depending on the type of the resin to be used, the exposure conditions, the required sensitivity, and the like. This makes it possible to exhibit superior sensitivity and LWR performance when forming a resist pattern.

### <Radiation-Sensitive Acid Generator>

The onium salt as the radiation-sensitive acid generator is a compound which contains an organic acid anion moiety (2) different from the organic acid anion moiety (1) and the onium cation moiety and generates an acid having a pKa lower than that of an acid generated from the acid diffusion controlling agent by irradiation with radioactive-ray. As in the case of the acid diffusion controlling agent, the onium cation moiety may be a fluorine-containing onium cation moiety or an onium cation moiety having no fluorine atom, but is preferably a fluorine-containing onium cation moiety. A part of the radiation-sensitive acid generator may contain a fluorine-containing onium cation moiety, and the remainder may contain an onium cation moiety having no fluorine atom.

The organic acid anion moiety (2) of the radiation-sensitive acid generator preferably has at least one selected from the group consisting of a sulfonate anion and a sulfonimide anion. The onium cation moiety is preferably at least one selected from the group consisting of a sulfonium cation and an iodonium cation. When the onium salt has a combination of these structures, the functions can be efficiently exhibited.

Examples of the acid generated by exposure to light may include acids that produce a sulfonic acid and a sulfonimide by exposure to light in response to the organic acid anion moiety (2). Among them, as the radiation-sensitive acid generator, a compound in which one or more fluorine atoms or fluorinated hydrocarbon groups are bonded to a carbon atom adjacent to a sulfur atom of a sulfonate anion as an onium salt that affords sulfonic acid by exposure to light is preferable.

The radiation-sensitive acid generator is preferably represented by the following formula (p-1).

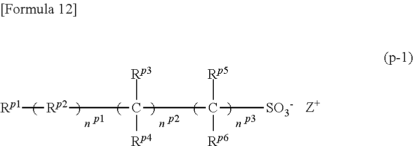

In the formula (p-1), R^{p1} is a monovalent group containing a ring structure having 6 or more ring members.

R^{p2} is a divalent linking group.

R^{p3} and R^{p4} are each independently a hydrogen atom, a fluorine atom, a monovalent hydrocarbon group having 1 to 20 carbon atoms, or a monovalent fluorinated hydrocarbon group having 1 to 20 carbon atoms.

R^{p5} and R^{p6} are each independently a fluorine atom or a monovalent fluorinated hydrocarbon group having 1 to 20 carbon atoms.

n^{p1} is an integer of 0 to 10, n^{p2} is an integer of 0 to 10, and n^{p3} is an integer of 0 to 10, provided that when n^{p1}+n^{p2}+n^{p3} is 0, R^{p1} has an aromatic ring structure as the ring structure, and the aromatic ring structure is bonded to —SO₃⁻ in the formula.

When n^{p1} is 2 or more, a plurality of R^{p2}s are identical to or different from each other.

When n^{p2} is 2 or more, a plurality of R^{p3}s are identical to or different from each other, and a plurality of R^{p4}s are identical to or different from each other.

When n^{p3} is 2 or more, a plurality of R^{p5}s are identical or different, and a plurality of R^{p6}s are identical or different.

Z⁺ is a monovalent onium cation.

Examples of the monovalent group containing a ring structure represented by R^{p1} may include a monovalent group containing an alicyclic structure having 5 or more ring members, a monovalent group containing an aliphatic heterocyclic structure having 5 or more ring members, a monovalent group containing an aromatic ring structure having 6 or more ring members, and a monovalent group containing an aromatic heterocyclic structure having 6 or more ring members, or a combination thereof.

Examples of the alicyclic structure having 5 or more ring members may include:
monocyclic cycloalkane structures such as a cyclopentane structure, a cyclohexane structure, a cycloheptane structure, a cyclooctane structure, a cyclononane structure, a cyclodecane structure, and a cyclododecane structure;
monocyclic cycloalkene structures such as a cyclopentene structure, a cyclohexene structure, a cycloheptene structure, a cyclooctene structure, and a cyclodecene structure;
polycyclic cycloalkane structures such as a norbornane structure, an adamantane structure, a tricyclodecane structure, and a tetracyclododecane structure; and
polycyclic cycloalkene structures such as a norbornene structure and a tricyclodecene structure.

Examples of the aliphatic heterocyclic structure having 5 or more ring members may include:
lactone structures such as a pentanolactone structure, a hexanolactone structure, and a norbornane lactone structure;
sultone structures such as a pentanosultone structure, a hexanosultone structure, and a norbornane sultone structure;
oxygen atom-containing heterocyclic structures such as an oxacyclopentane structure, an oxacycloheptane structure, and an oxanorbornane structure;
nitrogen atom-containing heterocyclic structures such as an azacyclopentane structure, an azacyclohexane structure, and a diazabicyclooctane structure; and
sulfur atom-containing heterocyclic structures of a thiacyclopentane structure, a thiacyclohexane structure, and a thianorbornane structure.

Examples of the aromatic ring structure having 6 or more ring members may include a benzene structure, a naphthalene structure, a phenanthrene structure, and an anthracene structure.

Examples of the aromatic heterocyclic structure having 6 or more ring members may include oxygen atom-containing heterocyclic structures such as a furan structure, a pyran structure, and a benzopyran structure, and nitrogen atom-containing heterocyclic structures such as a pyridine structure, a pyrimidine structure, and an indole structure.

The lower limit of the number of the ring members of the ring structure of R^{p1} may be 6, and is preferably 7, more preferably 8, still more preferably 9, particularly preferably 10. The upper limit of the number of the ring members is preferably 15, more preferably 14, still more preferably 13, particularly preferably 12.

Some or all of the hydrogen atoms of the ring structure of R^{p1} may be replaced with a substituent. Examples of the substituent may include halogen atoms such as a fluorine atom, a chlorine atom, a bromine atom, and an iodine atom, a hydroxy group, a carboxy group, a cyano group, a nitro group, an alkoxy group, an alkoxycarbonyl group, an alkoxycarbonyloxy group, an acyl group, and an acyloxy group. Among them, a hydroxy group is preferable.

Among them, as R^{p1}, monovalent groups containing an alicyclic structure having 5 or more ring members and monovalent groups containing an aliphatic heterocyclic structure having 5 or more ring members are preferable, monovalent groups containing an alicyclic structure having 6 or more ring members and monovalent groups containing an aliphatic heterocyclic structure having 6 or more ring members are more preferable, monovalent groups containing an alicyclic structure having 9 or more ring members and monovalent groups containing an aliphatic heterocyclic structure having 9 or more ring members are still more preferable, an adamantyl group, a hydroxyadamantyl group, a norbornane lactone-yl group, a norbornane sultone-yl group, and a 5-oxo-4-oxatricyclo[4.3.1.13,8]undecane-yl group are still more preferable, and an adamantyl group is particularly preferable.

Examples of the divalent linking group represented by R^{p2} may include a carbonyl group, an ether group, a carbonyloxy group, a sulfide group, a thiocarbonyl group, a sulfonyl group, a divalent hydrocarbon group, or a combination thereof. As the divalent linking group represented by R^{p2}, a carbonyloxy group, a sulfonyl group, an alkanediyl group, and a cycloalkanediyl group are preferable, a carbonyloxy group and a cycloalkanediyl group are more preferable, a carbonyloxy group and a norbornanediyl group are still more preferable, and a carbonyloxy group is particularly preferable.

Examples of the monovalent hydrocarbon group having 1 to 20 carbon atoms represented by R^{p3} and R^{p4} may include alkyl groups having 1 to 20 carbon atoms. Examples of the monovalent fluorinated hydrocarbon group having 1 to 20 carbon atoms represented by R^{p3} and R^{p4} may include fluorinated alkyl groups having 1 to 20 carbon atoms. As R^{p3} and R^{p4}, a hydrogen atom, a fluorine atom, and a fluorinated alkyl group are preferable, a fluorine atom and a perfluoroalkyl group are more preferable, and a fluorine atom and a trifluoromethyl group are still more preferable.

Examples of the monovalent fluorinated hydrocarbon group having 1 to 20 carbon atoms represented by R^{p5} and R^{p6} may include fluorinated alkyl groups having 1 to 20 carbon atoms. As R^{p5} and R^{p6}, a fluorine atom and a fluorinated alkyl group are preferable, a fluorine atom and a perfluoroalkyl group are more preferable, a fluorine atom and a trifluoromethyl group are still more preferable, and a fluorine atom is particularly preferable.

As n^{p1}, integers of 0 to 5 are preferable, integers of 0 to 3 are more preferable, integers of 0 to 2 are still more preferable, and 0 and 1 are particularly preferable.

As n^{p2}, integers of 0 to 2 are more preferable, 0 and 1 is still more preferable, and 0 is particularly preferable.

As n^{p3}, integers of 0 to 5 are preferable, integers of 1 to 4 are more preferable, integers of 1 to 3 are still more preferable, and 1 and 2 are particularly preferable. By adjusting n^{p3} to 1 or more, the strength of an acid generated from the compound of the formula (p-1) can be increased, and as a result, the LWR performance and the like of the radiation-sensitive resin composition can be further improved. The upper limit of n^{p3} is preferably 4, more preferably 3, still more preferably 2.

In the formula (p-1), n^{p1}+n^{p2}+n^{p3} is an integer of 0 or more and 30 or less. When n^{p1}+n^{p2}+n^{p3} is 0, R^{p1} has an aromatic ring structure as the ring structure, and the aromatic ring structure is bonded to —SO₃ in the formula (p-1). The lower limit of n^{p1}+n^{p2}+n^{p3} is preferably 2, more preferably 4. The upper limit of n^{p1}+n^{p2}+n^{p3} is preferably 20, more preferably 10.

As the monovalent onium cation represented by Z⁺, an onium cation moiety of the acid diffusion controlling agent can be suitably employed.

Examples of the radiation-sensitive acid generator represented by the formula (p-1) include radiation-sensitive acid generators represented by the following formulas (p-1-1) to (p-1-40) (hereinafter, also referred to as “a radiation-sensitive acid generator (p-1-1) to a radiation-sensitive acid generator (p-1-40)”).

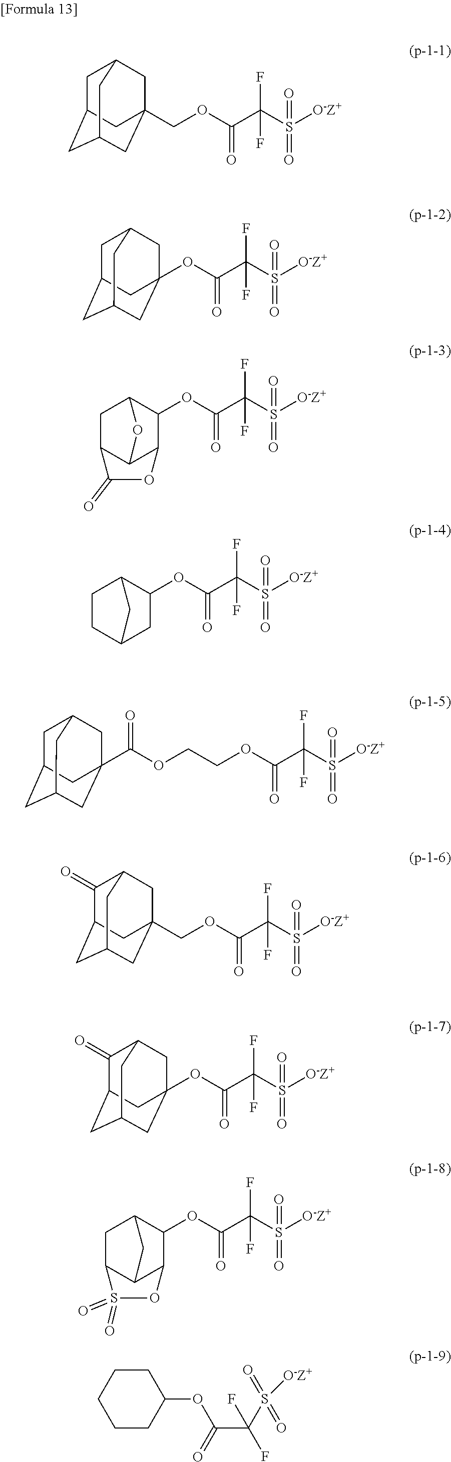

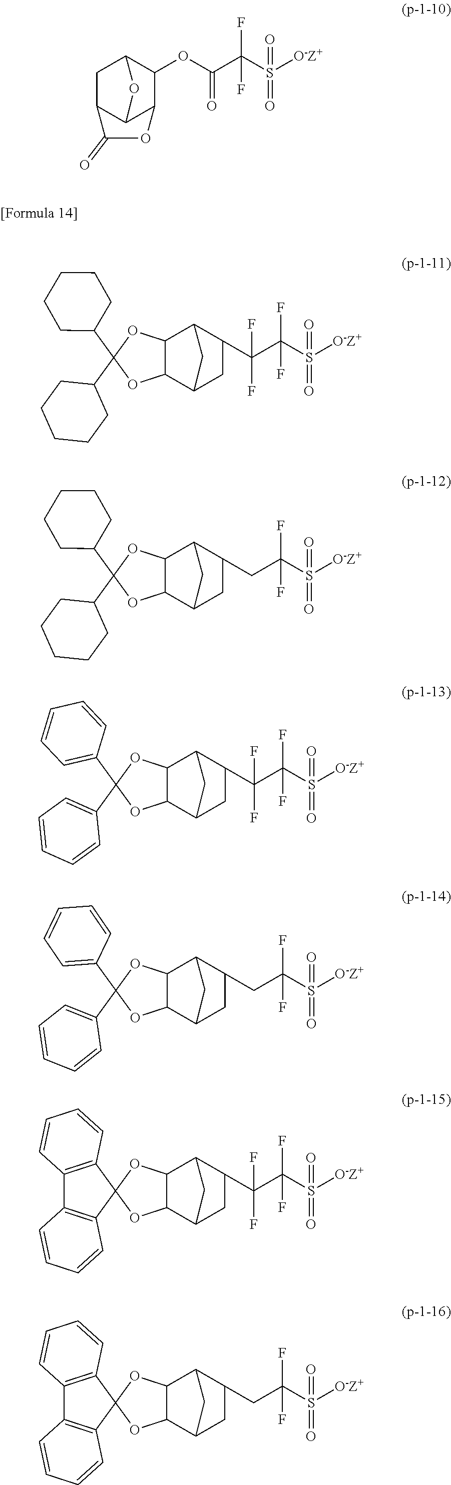

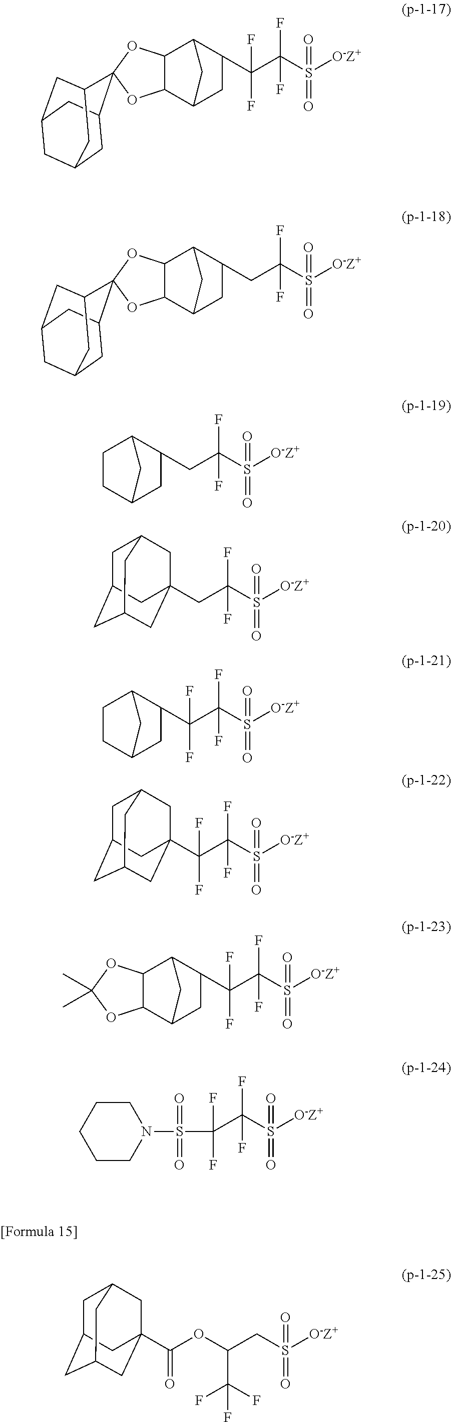

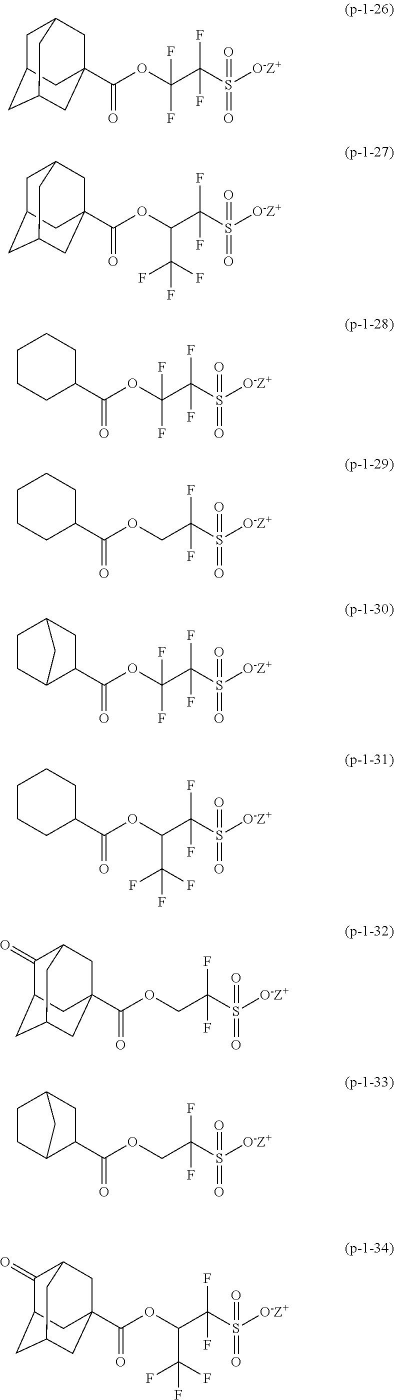

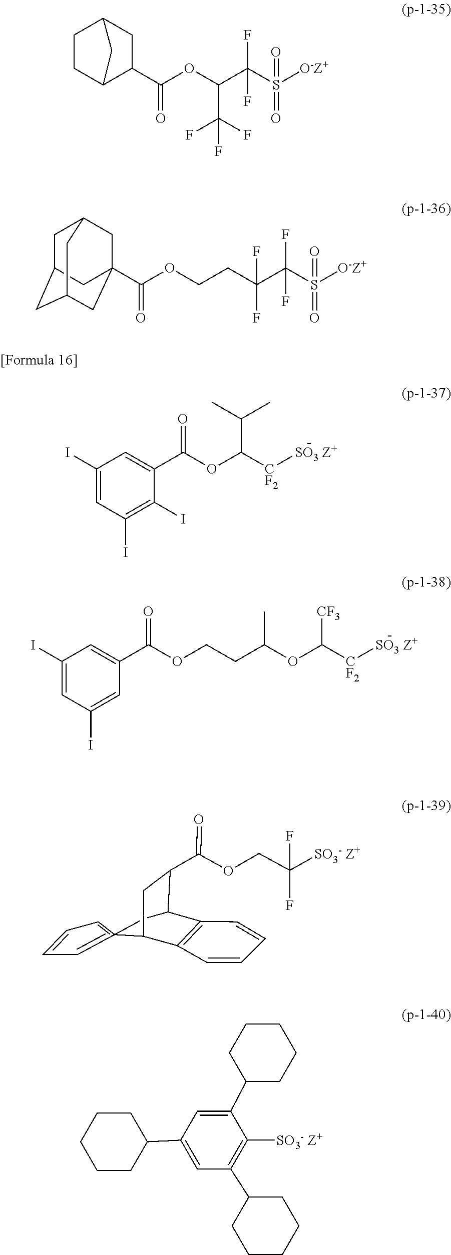

In the formulas (p-1-1) to (p-1-40), Z⁺ is a monovalent onium cation.

A method for synthesizing the radiation-sensitive acid generator represented by the formula (p-1) can also be synthesized by a known method, particularly by a salt exchange reaction. Another known radiation-sensitive acid generator can also be used in combination as long as the effects of the present invention are not impaired.

The radiation-sensitive acid generators may be used singly, or two or more thereof may be used in combination. The lower limit of the content of the radiation-sensitive acid generator is preferably 5 parts by mass, more preferably 10 parts by mass, still more preferably 15 parts by mass, with respect to 100 parts by mass of the resin described later. The upper limit of the content is preferably 40 parts by mass, more preferably 30 parts by mass, even more preferably 25 parts by mass. This makes it possible to exhibit superior sensitivity and LWR performance when forming a resist pattern.

### <Resin>

The resin is an assembly of polymers containing a structural unit having an acid-dissociable group (hereinafter, also referred to as “structural unit (I)”) (this resin is hereinafter also referred to as “base resin”). The base resin may contain, in addition to the structural unit (I), a structural unit having a phenolic hydroxyl group (II), a structural unit containing a heteroatom-containing substituent or the like (III), a structural unit containing a lactone structure or the like (IV), or the like. Hereinafter, each of the structural units will be described.

### (Structural Unit (I))

The structural unit (I) is a structural unit having an acid-dissociable group. In the present description, the “acid-dissociable group” refers to a group that substitutes for a hydrogen atom of alkali-soluble groups such as a carboxy group, a phenolic hydroxyl group, a sulfo group, and a sulfonamide group, and is dissociated by the action of an acid. Therefore, the acid-dissociable group is bonded to an oxygen atom that would otherwise be bonded to the hydrogen atom in these functional groups.

The structural unit (I) is not particularly limited as long as the structural unit (I) has an acid-dissociable group, and examples thereof include a structural unit having a tertiary alkyl ester moiety, a structural unit having a structure in which a hydrogen atom of a phenolic hydroxy group is substituted with a tertiary alkyl group. Among them, a structural unit represented by the following formula (3) (hereinafter, also referred to as “structural unit (1-1)”) is preferable from the viewpoint of improvement in the patternability of the radiation-sensitive resin composition.

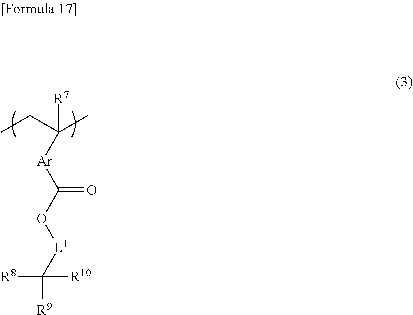

In the formula (3), R⁷ is a hydrogen atom, a fluorine atom, a methyl group, or a trifluoromethyl group. R⁸ is a hydrogen atom or a monovalent hydrocarbon group having 1 to 20 carbon atoms. R⁹ and R¹⁰ are each independently a monovalent chain hydrocarbon group having 1 to 10 carbon atoms or a monovalent alicyclic hydrocarbon group having 3 to 20 carbon atoms, or represent a divalent alicyclic group having 3 to 20 carbon atoms composed of R⁹ and R¹⁰ combined with each other together with a carbon atom to which R⁹ and R¹⁰ are bonded. Ar is a single bond or a substituted or unsubstituted phenylene group. L¹ represents a single bond or a divalent linking group.

As R⁷, from the viewpoint of the copolymerizability of a monomer that affords the structural unit (1-1), a hydrogen atom and a methyl group are preferable, and a methyl group is more preferable.

Examples of the monovalent hydrocarbon group having 1 to 20 carbon atoms represented by R⁸ include a chain hydrocarbon group having 1 to 10 carbon atoms, a monovalent alicyclic hydrocarbon group having 3 to 20 carbon atoms, and a monovalent aromatic hydrocarbon group having 6 to 20 carbon atoms.

Examples of the chain hydrocarbon groups having 1 to 10 carbon atoms represented by R⁸ to R¹⁰ include a linear or branched saturated hydrocarbon group having 1 to 10 carbon atoms or a linear or branched unsaturated hydrocarbon group having 1 to 10 carbon atoms.

Examples of the alicyclic hydrocarbon groups having 3 to 20 carbon atoms represented by R⁸ to R¹⁰ include monocyclic or polycyclic saturated hydrocarbon groups and monocyclic or polycyclic unsaturated hydrocarbon groups. As the monocyclic saturated hydrocarbon group, a cyclopentyl group, a cyclohexyl group, a cycloheptyl group, and a cyclooctyl group are preferable. As the polycyclic cycloalkyl group, bridged alicyclic hydrocarbon groups such as a norbornyl group, an adamantyl group, a tricyclodecyl group, and a tetracyclododecyl group are preferable. The bridged alicyclic hydrocarbon group refers to a polycyclic alicyclic hydrocarbon group in which two carbon atoms among carbon atoms composing an alicyclic ring that are not adjacent to each other are bonded by a bonding chain containing one or more carbon atoms.

Examples of the monovalent aromatic hydrocarbon group having 6 to 20 carbon atoms represented by R⁸ include aryl groups such as a phenyl group, a tolyl group, a xylyl group, a naphthyl group, and an anthryl group; and aralkyl groups such as a benzyl group, a phenethyl group, and a naphthylmethyl group.

As R⁸, a linear or branched saturated hydrocarbon group having 1 to 10 carbon atoms, an alicyclic hydrocarbon group having 3 to 20 carbon atoms, and a monovalent aromatic hydrocarbon group having 6 to 10 carbon atoms are preferable.

The divalent alicyclic group having 3 to 20 carbon atoms composed of R⁹ and R¹⁰ combined with each other together with a carbon atom to which R⁹ and R¹⁰ are bonded is not particularly limited, as long as the divalent alicyclic group is a group obtained by removing two hydrogen atoms from an identical carbon atom composing a carbon ring of a monocyclic or polycyclic alicyclic hydrocarbon having the aforementioned number of carbon atoms. The divalent alicyclic group may be either a monocyclic hydrocarbon group or a polycyclic hydrocarbon group, and the polycyclic hydrocarbon group may be either a bridged alicyclic hydrocarbon group or a fused alicyclic hydrocarbon group, and may be either a saturated hydrocarbon group or an unsaturated hydrocarbon group. The fused alicyclic hydrocarbon group refers to a polycyclic alicyclic hydrocarbon group composed of a plurality of alicyclic rings sharing a side (a bond between two adjacent carbon atoms).

Among the monocyclic alicyclic hydrocarbon groups, as the saturated hydrocarbon group, a cyclopentanediyl group, a cyclohexanediyl group, a cycloheptanediyl group, a cyclooctanediyl group, and the like are preferable, and as the unsaturated hydrocarbon group, a cyclopentenediyl group, a cyclohexenediyl group, a cycloheptenediyl group, a cyclooctenediyl group, a cyclodecenediyl group, and the like are preferable. As the polycyclic alicyclic hydrocarbon group, bridged alicyclic saturated hydrocarbon groups are preferable, and for example, a bicyclo[2.2.1]heptane-2,2-diyl group (norbornane-2,2-diyl group), a bicyclo[2.2.2]octane-2,2-diyl group, and a tricyclo[3.3.1.1^{3,7}]decane-2,2-diyl group (adamantane-2,2-diyl group) are preferable.

The phenylene group represented by Ar may be any of a 1,4-phenylene group, a 1,3-phenylene group, and a 1,2-phenylene group. When the phenylene group has a substituent, as the substituent, a group represented by R² in the formula (1) can be suitably employed.

Examples of the divalent linking group represented by L¹ include an alkanediyl group, a cycloalkanediyl group, an alkenediyl group, *—R^{LA}O—, and *-R^{LB}COO— (* represents a bond with an oxygen atom), provided that in the case of a group other than *—R^{LB}COO—, the carbon atom bonded to the oxygen atom of —COO— in the formula (3) is a tertiary carbon and has no hydrogen atom.

Some or all of the hydrogen atoms on the carbon atom in R⁸ to R¹⁰ and L¹ may be substituted with a halogen atom such as a fluorine atom, a chlorine atom, or an iodine atom, a halogenated alkyl group such as a trifluoromethyl group, an alkoxy group such as a methoxy group, a cyano group, or the like.

The alkanediyl group is preferably an alkanediyl group having 1 to 8 carbon atoms.

Examples of the cycloalkanediyl group include monocyclic cycloalkanediyl groups such as a cyclopentanediyl group and a cyclohexanediyl group; and polycyclic cycloalkanediyl groups such as a norbornanediyl group and an adamantanediyl group. The cycloalkanediyl group is preferably a cycloalkanediyl group having 5 to 12 carbon atoms.

Examples of the alkenediyl group include an ethenediyl group, a propenediyl group, and a butenediyl group. The alkenediyl group is preferably an alkenediyl group having 2 to 6 carbon atoms.

Examples of R^{LA} of *—R^{LA}O— include the alkanediyl group, the cycloalkanediyl group, and the alkenediyl group. Examples of R^{LB} of *—R^{LB}COO— include the alkanediyl group, the cycloalkanediyl group, the alkenediyl group, and an arenediyl group. Examples of the arenediyl group include a phenylene group, a tolylene group, and a naphthylene group. The arenediyl group is preferably an arenediyl group having 6 to 15 carbon atoms.

Among them, it is preferable that R⁸ be an alkyl group having 1 to 4 carbon atoms or an aryl group having 6 to 10 carbon atoms, and the alicyclic structure composed of R⁹ and R¹⁰ combined with each other together with a carbon atom to which R⁹ and R¹⁰ are bonded be a polycyclic or monocyclic cycloalkane structure. L¹ is preferably a single bond or *—R^{LA}O—. As R^{LA}, an alkanediyl group is preferable.

Examples of the structural unit (1-1) include structural units represented by the following formulas (3-1) to (3-8) (hereinafter, also referred to as “structural units (1-1-1) to (1-1-8)”).

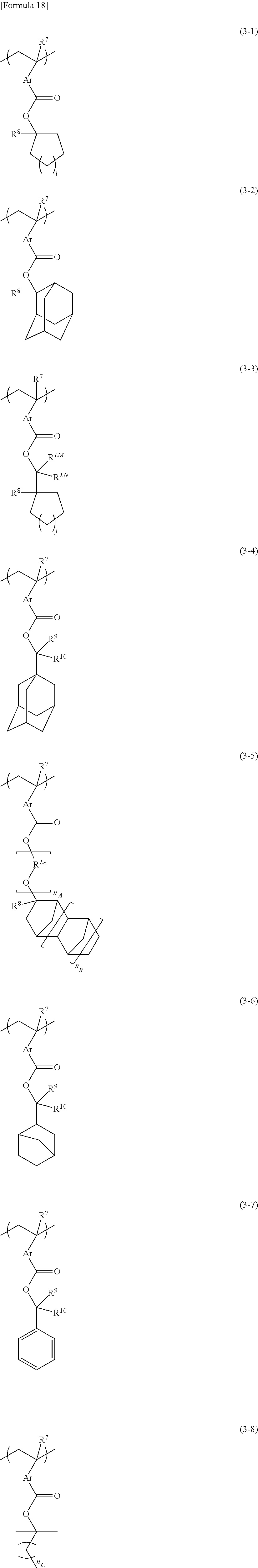

In the formulas (3-1) to (3-8), R⁷ to R¹⁰, R^{LA}, and Ar have the same meaning as in the formula (3) R^{LM} and R^{LN} are each independently a monovalent hydrocarbon group having 1 to 10 carbon atoms, i and j are each independently an integer of 1 to 4. n_{A}, n_{B}, and n_{C} are each independently 0 or 1.

Examples of R^{LM} and R^{LN} include groups corresponding to the monovalent hydrocarbon groups having 1 to 10 carbon atoms among the monovalent hydrocarbon groups having 1 to 20 carbon atoms represented by R⁸ of the formula (3). As R^{LM} and R^{LN}, a methyl group, an ethyl group, or an isopropyl group are preferable.

i and j are preferably 1, 2, or 4. As R⁸ to R¹⁰, a methyl group, an ethyl group, an isopropyl group, or a phenyl group is preferable.

The base resin may contain one type of the structural unit (I) or two or more types of the structural unit (I) in combination.

Furthermore, the resin may contain, as the structural unit (I), a structural unit represented by the following formulas (1f) to (2f) together with or in place of the structural unit (1-1).

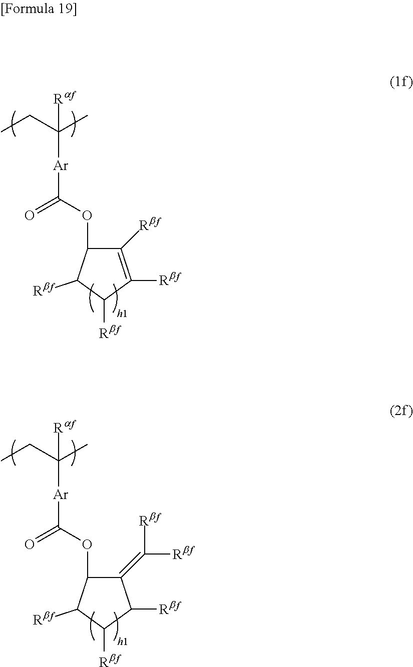

In the formulas (1f) to (2f), R^{αf}s are each independently a hydrogen atom, a fluorine atom, a methyl group, or a trifluoromethyl group. R^{βf}s are each independently a hydrogen atom or a chain alkyl group having 1 to 5 carbon atoms. Ar has the same meaning as in the formula (3). h₁s are each independently an integer of 1 to 4.

As the R^{βf}, a hydrogen atom, a methyl group, or an ethyl group is preferable. h₁ is preferably 1 or 2.

The lower limit of the content ratio of the structural unit (I) is preferably 20 mol %, more preferably 30 mol %, still more preferably 40 mol %, particularly preferably 50 mol % with respect to all the structural units composing the base resin. The upper limit of the content ratio is preferably 90 mol %, more preferably 80 mol %, still more preferably 70 mol %, particularly preferably 65 mol %. When the content ratio of the structural unit (I) is adjusted to within the above range, the patternability of the radiation-sensitive resin composition can be further improved.

### (Structural Unit (II))

The structural unit (II) is a structural unit having a phenolic hydroxyl group (provided that a case where the structural unit corresponds to the structural unit (I) is excluded). In the present invention, a phenolic hydroxy group generated through deprotection due to the action of an acid generated by exposure to light is also included as the phenolic hydroxy group of the structural unit (II). When the resin contains the structural unit (II), the solubility in a developer can be more appropriately adjusted, and as a result, the sensitivity and the like of the radiation-sensitive resin composition can be further improved. When KrF excimer laser light, EUV, electron beam or the like is used as radioactive-ray to be applied in an exposure step in a resist pattern formation method, the structural unit (II) contributes to improvement in etching resistance and improvement in difference in solubility in a developer between an exposed area and an unexposed area (dissolution contrast). In particular, the polymer containing the structural unit (III) can be suitably applied for pattern formation using exposure with radiation having a wavelength of 50 nm or less, such as an electron beam or EUV. The structural unit (II) is preferably represented by the following formula (2),

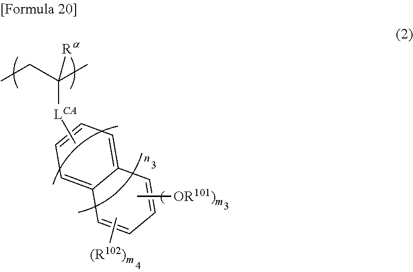

(In the formula (2),

R^{α} is a hydrogen atom, a fluorine atom, a methyl group, or a trifluoromethyl group.

L^{CA} is a single bond, —COO—*, or —O—*. * is a bond on the aromatic ring side.

R¹⁰¹ is a hydrogen atom or a protective group to be deprotected by the action of an acid. When there are a plurality of R¹⁰¹s, the plurality of R¹⁰¹s are the same as or different from each other.

R¹⁰² is a cyano group, a nitro group, an alkyl group, a fluorinated alkyl group, an alkoxycarbonyloxy group, an acyl group, or an acyloxy group. When there are a plurality of R¹⁰²s, the plurality of R¹⁰²s are the same as or different from each other.

n₃ is an integer of 0 to 2, m₃ is an integer of 1 to 8, and m₄ is an integer of 0 to 8, provided that 1 m₃+m₄+m₅ 2n3+5 is satisfied.)

As R^{α}, a hydrogen atom or a methyl group is preferable from the viewpoint of the copolymerizability of a monomer that affords the structural unit C.

L^{CA} is preferably a single bond or —COO—*.

Examples of the protecting group to be deprotected by the action of an acid represented by R¹⁰¹ include groups represented by the following formulas (AL-1) to (AL-3).

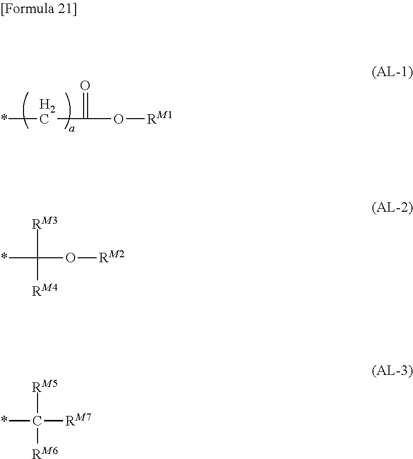

In the formulas (AL-1) and (AL-2), R^{M1} and R^{M2} are monovalent hydrocarbon groups, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 40 carbon atoms, more preferably an alkyl group having 1 to 20 carbon atoms. In the formula (AL-1), a is an integer of 0 to 10, preferably an integer of 1 to 5. In the formulas (AL-1) to (AL-3), * is a bond to another moiety.

In the formula (AL-2), R^{M3} and R^{M4} are each independently a hydrogen atom or a monovalent hydrocarbon group, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M2}, R^{M3}, and R^{M4} may be bonded to each other to form a ring having 3 to 20 carbon atoms together with a carbon atom or a carbon atom and an oxygen atom to which two thereof are bonded. The ring is preferably a ring having 4 to 16 carbon atoms, particularly preferably an alicyclic ring.

In the formula (AL-3), R^{M5}, R^{M6}, and R^{M7} are each independently a monovalent hydrocarbon group, and may contain a hetero atom such as an oxygen atom, a sulfur atom, a nitrogen atom, or a fluorine atom. The monovalent hydrocarbon group may be linear, branched, or cyclic, and is preferably an alkyl group having 1 to 20 carbon atoms. Any two of R^{M5}, R^{M6}, and R^{M7} may be bonded to each other to form a ring having 3 to 20 carbon atoms together with a carbon atom to which two thereof are bonded. The ring is preferably a ring having 4 to 16 carbon atoms, particularly preferably an alicyclic ring.

Among them, the protective group to be deprotected by the action of an acid is preferably a group represented by the formula (AL-3).

Examples of the alkyl group in R¹⁰² include linear or branched alkyl groups having 1 to 8 carbon atoms such as a methyl group, an ethyl group, and a propyl group. Examples of the fluorinated alkyl group include linear or branched fluorinated alkyl groups having 1 to 8 carbon atoms such as a trifluoromethyl group and a pentafluoroethyl group. Examples of the alkoxycarbonyloxy group include chain or alicyclic alkoxycarbonyloxy groups having 2 to 16 carbon atoms such as a methoxycarbonyloxy group, a butoxycarbonyloxy group, and an adamantylmethyloxycarbonyloxy group. Examples of the acyl group include aliphatic or aromatic acyl groups having 2 to 12 carbon atoms such as an acetyl group, a propionyl group, a benzoyl group, and an acryloyl group. Examples of the acyloxy group include aliphatic or aromatic acyloxy groups having 2 to 12 carbon atoms such as an acetyloxy group, a propionyloxy group, a benzoyloxy group, and an acryloyloxy group.

The n₃ is more preferably 0 or 1, still more preferably 0.

m₃ is preferably an integer of 1 to 3, more preferably 1 or 2.

m₄ is preferably an integer of 0 to 3, more preferably an integer of 0 to 2.

As the structural unit (II), structural units represented by the following formulas (2-1) to (2-10) (hereinafter, also referred to as “structural units (2-1) to (2-10)”) and the like are preferable.

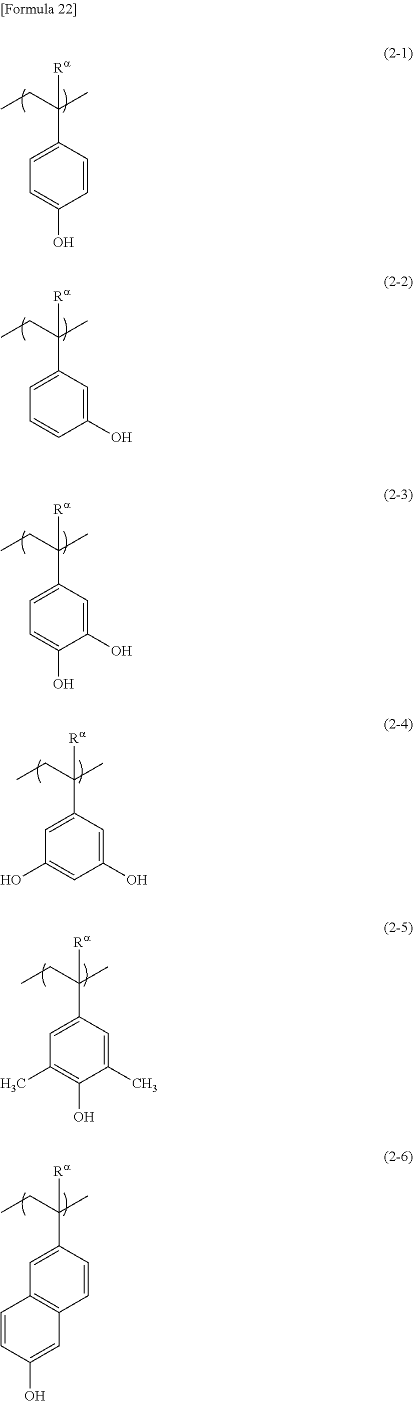

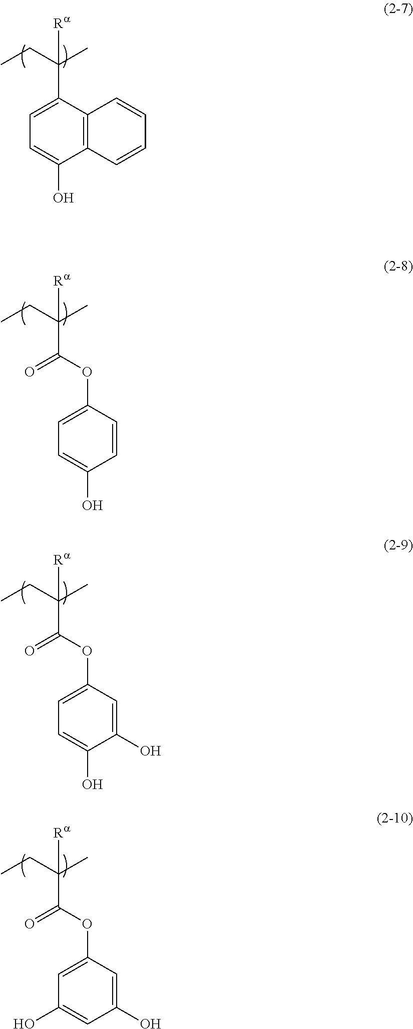

In the formulas (2-1) to (2-10), R^{a} is the same as in the formula (2).

Among them, the structural units (2-1) to (2-4), (2-6), (2-8), and (2-9) are preferable.

The lower limit of the content ratio of the structural unit (II) (the total when there are a plurality of types of the structural unit (II)) is preferably 10 mol %, more preferably 15 mol %, still more preferably 20 mol %, particularly preferably 25 mol % with respect to all the structural units composing the resin. The upper limit of the content ratio is preferably 60 mol %, more preferably 55 mol %, still more preferably 50 mol %, particularly preferably 45 mol %. When the content ratio of the structural unit (II) is adjusted to within the above range, the sensitivity, LWR performance, and resolution of the radiation-sensitive resin composition can be further improved.

In the case of polymerizing a monomer having a phenolic hydroxy group such as hydroxystyrene, it is preferable to polymerize the monomer with the phenolic hydroxy group protected by a protecting group such as an alkali-dissociable group and then perform deprotection by hydrolysis to yield a structural unit (II). The monomer may be polymerized without protecting the phenolic hydroxyl group.

### (Structural Unit (III))

The structural unit (III) is a structural unit (excluding structures corresponding to the structural units (I) to (II)) having a heteroatom-containing substituent such as a fluorine atom, an alcoholic hydroxyl group, a carboxy group, a cyano group, a nitro group, or a sulfonamide group. Among them, a structural unit having a fluorine atom, a structural unit having an alcoholic hydroxyl group, and a structural unit having a carboxy group are preferable, and a structural unit having a fluorine atom, and a structural unit having an alcoholic hydroxyl group are more preferable.

Examples of the structural unit (III) include structural units represented by the following formulas.

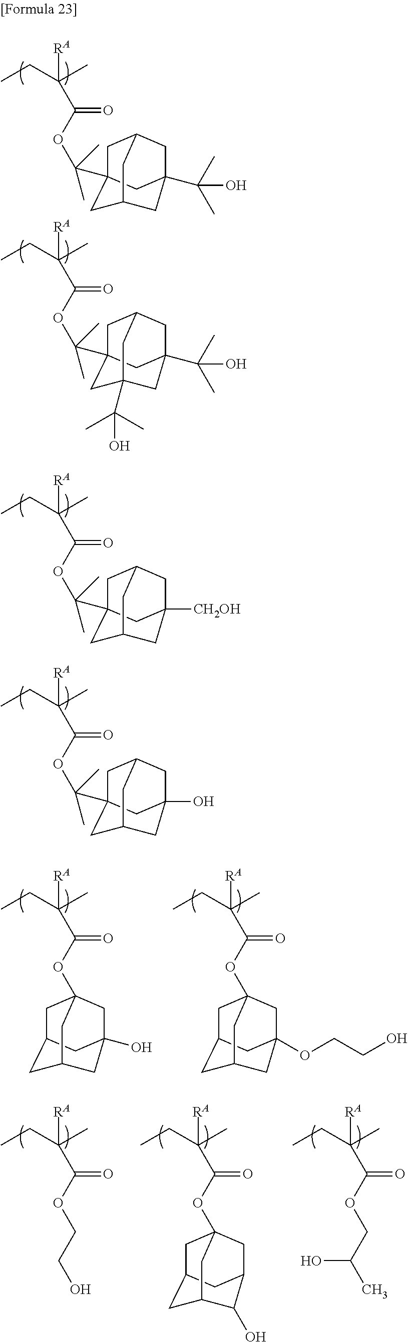

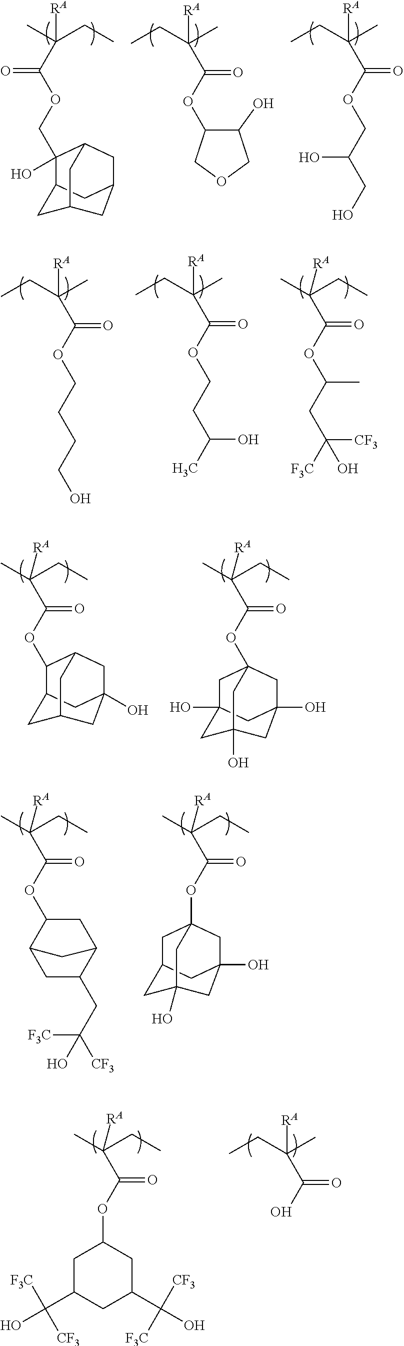

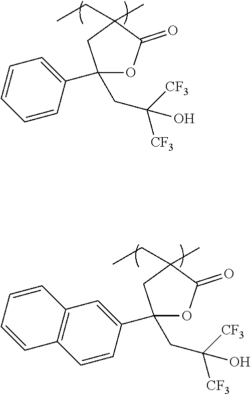

In the formulas, R^{A} is a hydrogen atom, a fluorine atom, a methyl group, or a trifluoromethyl group.

When the resin contains the structural unit (III), the lower limit of the content ratio of the structural unit (III) is preferably 3 mol %, more preferably 5 mol %, still more preferably 8 mol % with respect to all the structural units composing the resin. The upper limit of the content ratio is preferably 20 mol %, more preferably 15 mol %, still more preferably 12 mol %. When the content ratio of the structural unit (III) is adjusted to within the above range, the solubility of the resin in a developer can be made more appropriate.

### (Structural Unit (IV))

The structural unit (IV) is a structural unit containing at least one selected from the group consisting of a lactone structure, a cyclic carbonate structure, and a sultone structure. When the base resin further has the structural unit (IV), the solubility in a developer can be adjusted, and as a result, the lithographic performance, such as resolution, of the radiation-sensitive resin composition can be improved. In addition, the adhesion between a resist pattern formed of the base resin and a substrate can be improved.

### (Method for Synthesizing Resin)

A resin as the base resin can be synthesized by, for example, subjecting monomers that afford respective structural units to a polymerization reaction in an appropriate solvent using a publicly known radical polymerization initiator and the like.

The molecular weight of a resin as the base resin is not particularly limited, but the lower limit of the weight-average molecular weight (Mw) in terms of polystyrene as determined by Gel Permeation Chromatography (GPC) is preferably 1,000, more preferably 2,000, still more preferably 3,000, particularly preferably 4,000. The upper limit of the Mw is preferably 30,000, more preferably 20,000, still more preferably 10,000, particularly preferably 8,000. When the Mw of the resin is within the above range, an obtained resist film is good in heat resistance and developability.

The ratio of Mw to the number-average molecular weight (Mn) (Mw/Mn) of the resin as a base resin in terms of polystyrene as determined by GPC is usually 1 or more and 5 or less, preferably 1 or more and 3 or less, more preferably 1 or more and 2 or less.

The method for measuring Mw and Mn of a resin in the present description is as described in Examples.

The content of the resin is preferably 70 mass % or more, more preferably 75 mass % or more, still more preferably 80 mass % or more with respect to the total solid content of the radiation-sensitive resin composition.

### (Structural Unit (V))

The resin may contain a structural unit having an organic acid anion moiety and an onium cation moiety as an additional structural unit (V). When the resin contains the structural unit (V), the resin can function as the acid generator or the acid diffusion controlling agent. In this case, the composition may contain the radiation-sensitive acid generator or the acid diffusion controlling agent, or otherwise. The structural unit (V) functioning as the acid generator is preferably represented by the following formula (a1) or (a2).

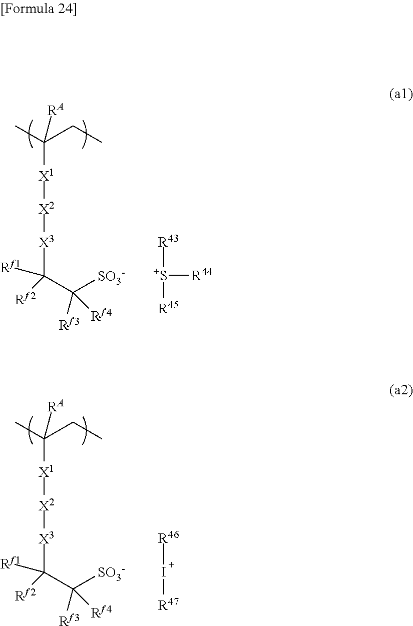

In the formulas, R^{A} is a hydrogen atom or a methyl group. X¹ is a single bond or an ester group. X² is a linear, branched or cyclic alkylene group having 1 to 12 carbon atoms or an arylene group having 6 to 10 carbon atoms, and some of the methylene groups composing the alkylene group may be substituted with an ether group, an ester group, or a lactone ring-containing group. At least one hydrogen atom contained in X² may be substituted with an iodine atom. X³ is a single bond, an ether group, an ester group, or a linear, branched or cyclic alkylene group having 1 to 12 carbon atoms, and some of the methylene groups composing the alkylene group may be substituted with an ether group or an ester group. Rf¹ to Rf⁴ are each independently a hydrogen atom, a fluorine atom, or a trifluoromethyl group, and at least one of Rf¹ to Rf⁴ is a fluorine atom or a fluorinated hydrocarbon group. R⁴³ to R⁴⁷ are each independently a monovalent hydrocarbon group having 1 to 20 carbon atoms and optionally containing a heteroatom, and R⁴³ and R⁴⁴ may be bonded to each other to form a ring together with a sulfur atom to which R⁴³ and R⁴⁴ are bonded.

As the monovalent hydrocarbon group having 1 to 20 carbon atoms and optionally containing a heteroatom in R⁴³ to R⁴⁷, an alkyl group having 1 to 12 carbon atoms, a cycloalkyl group having 3 to 12 carbon atoms, or an aryl group having 6 to 20 carbon atoms is preferable, and some or all of the hydrogen atoms of these groups may be substituted with a hydroxy group, a carboxy group, a halogen atom, an oxo group, a cyano group, an amide group, a nitro group, a sultone group, a sulfone group, or a sulfonium salt-containing group, and some of the methylene groups composing these groups may be substituted with an ether group, an ester group, a carbonyl group, a carbonate group, or a sulfonate ester group.

The formulas (a1) and (a2) are preferably represented by the following formulas (a1-1) and (a2-1), respectively.

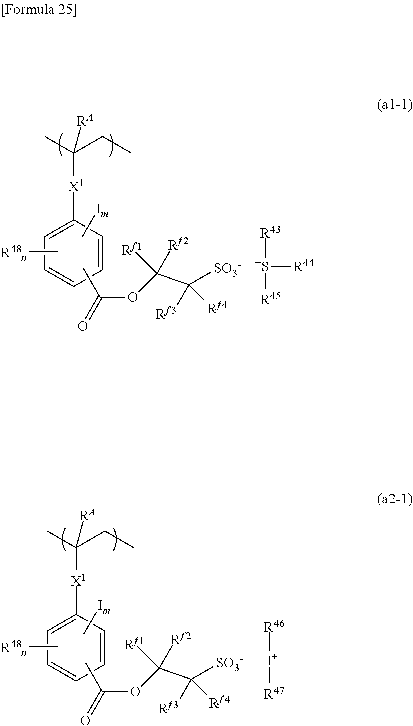

In the formulas, R^{A}, R⁴³ to R⁴⁷, Rf¹ to Rf⁴, and X¹ have the same meaning as in the formula (a1) or (a2). R⁴⁸ is a linear, branched or cyclic alkyl group having 1 to 4 carbon atoms, a halogen atom other than an iodine atom, a hydroxy group, a linear, branched or cyclic alkoxy group having 1 to 4 carbon atoms, or a linear, branched or cyclic alkoxycarbonyl group having 2 to 5 carbon atoms. m is an integer of 0 to 4. n is an integer of 0 to 3.

Examples of the organic acid anion moiety of the monomer that affords the structural unit (V) include, but are not limited to, those shown below. While all of those shown below are organic acid anion moieties having an iodine-substituted aromatic ring structure, as organic acid anion moieties having no iodine-substituted aromatic ring structure, structures in which the iodine atoms in the formulas shown below are substituted with an atom or a group other than an iodine atom such as a hydrogen atom or another substituent can be suitably employed.

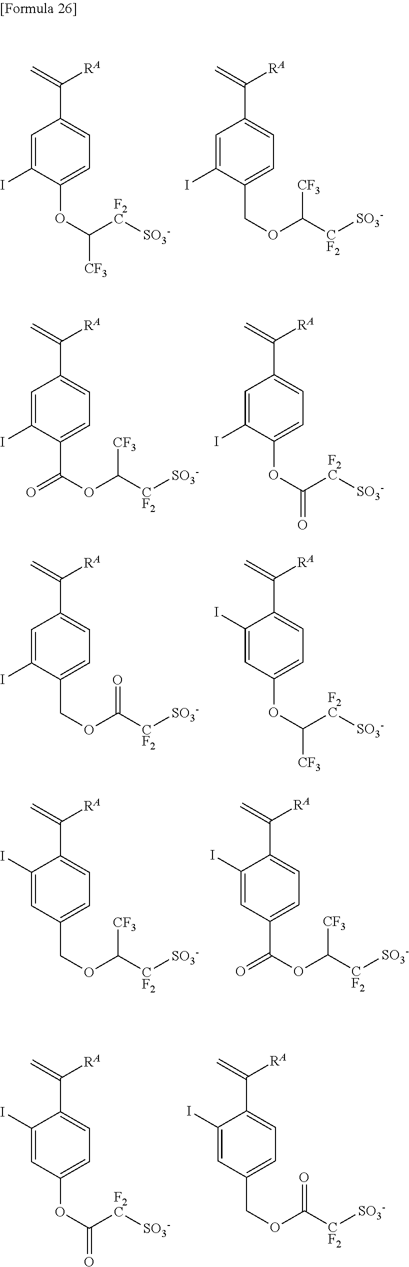

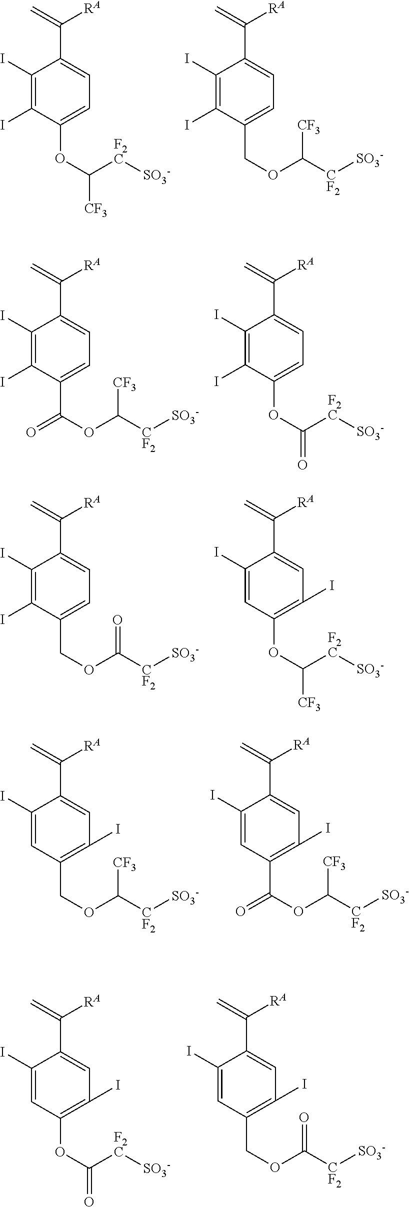

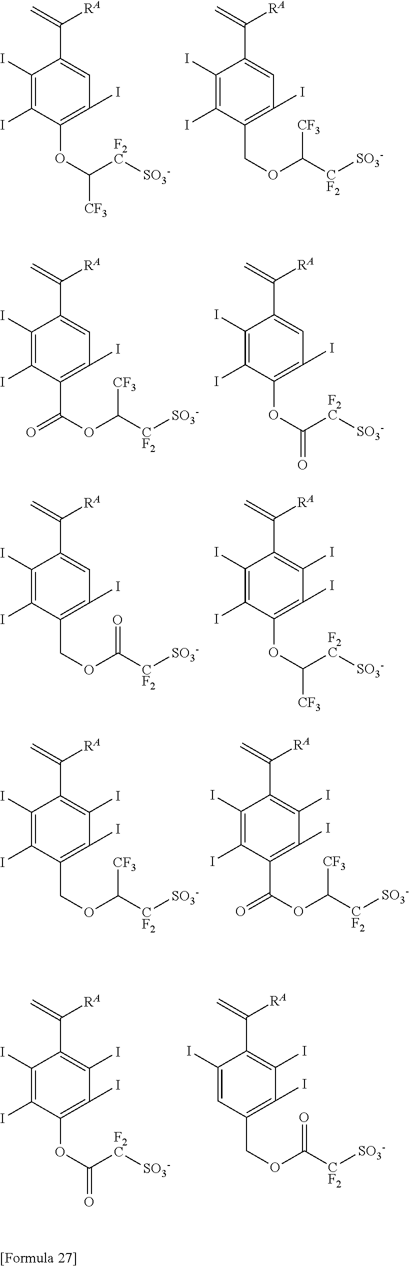

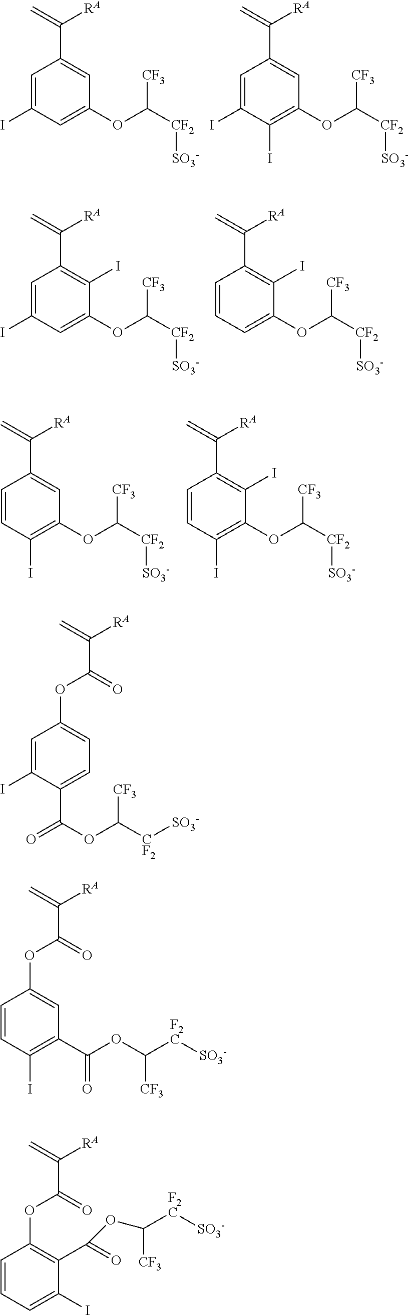

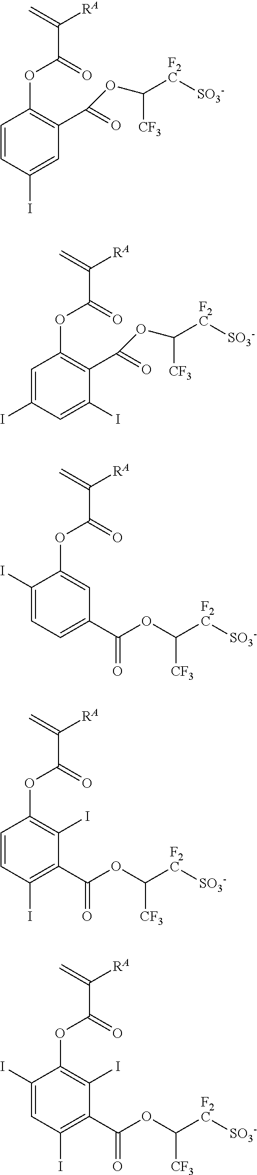

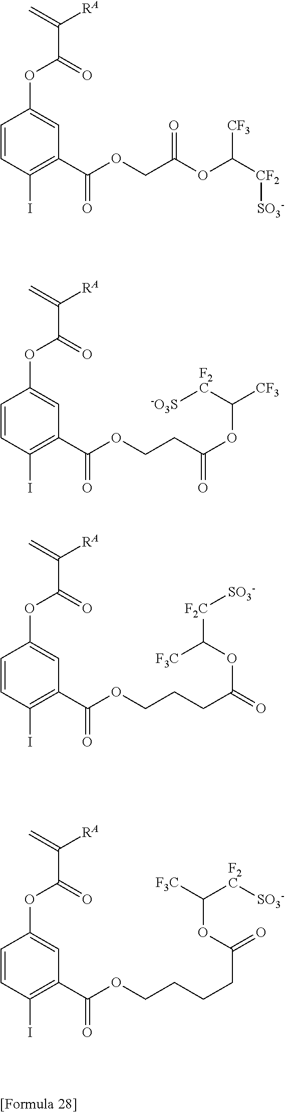

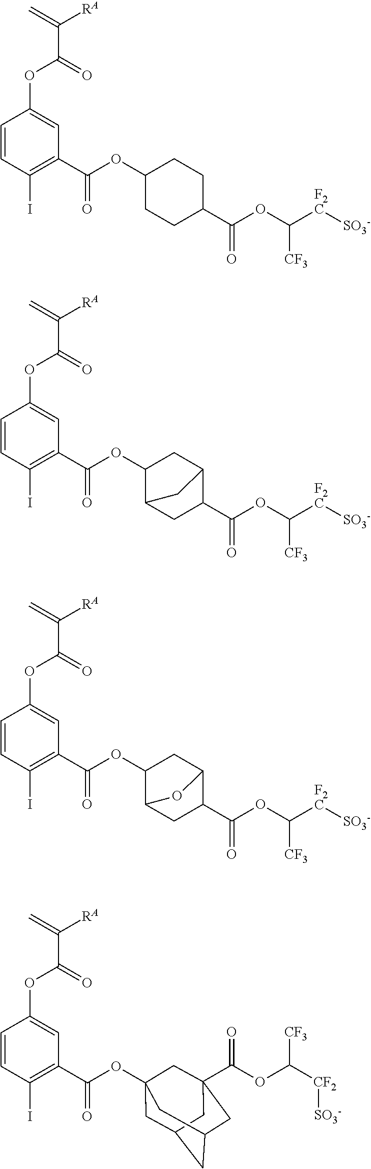

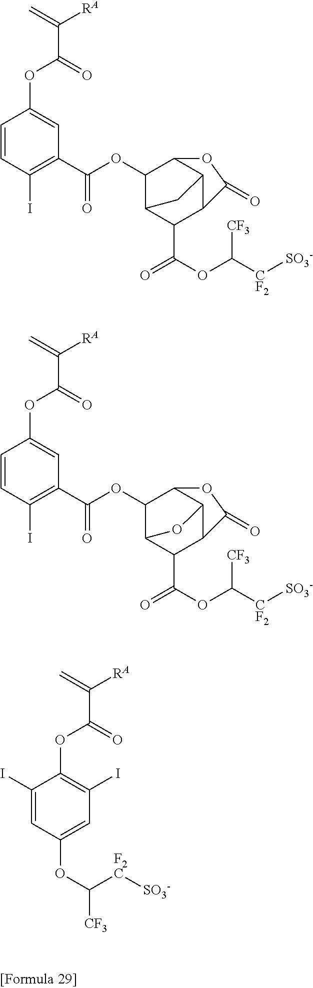

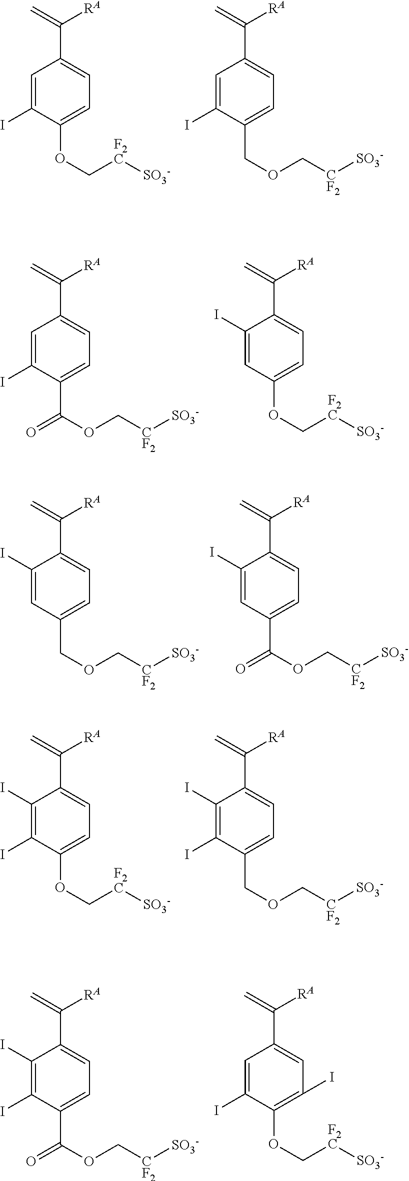

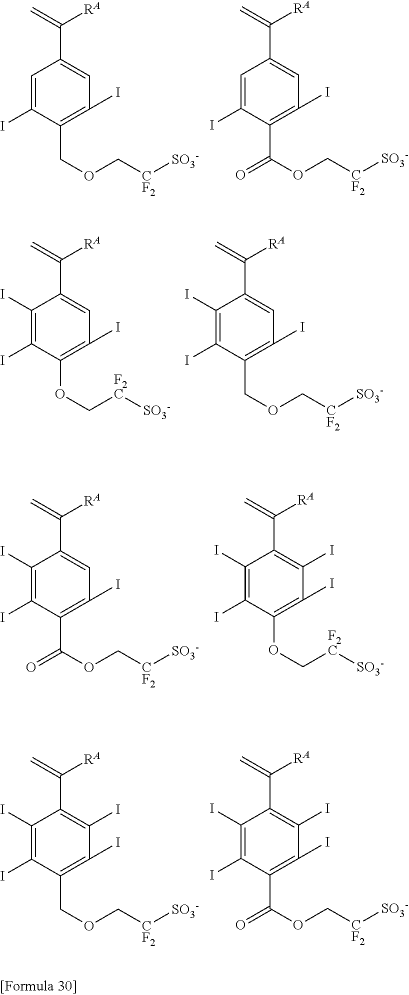

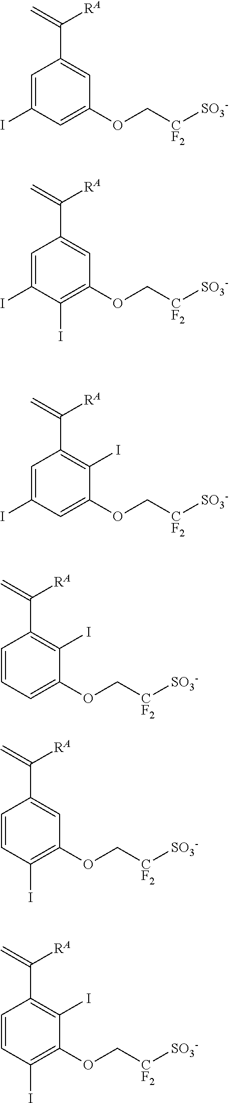

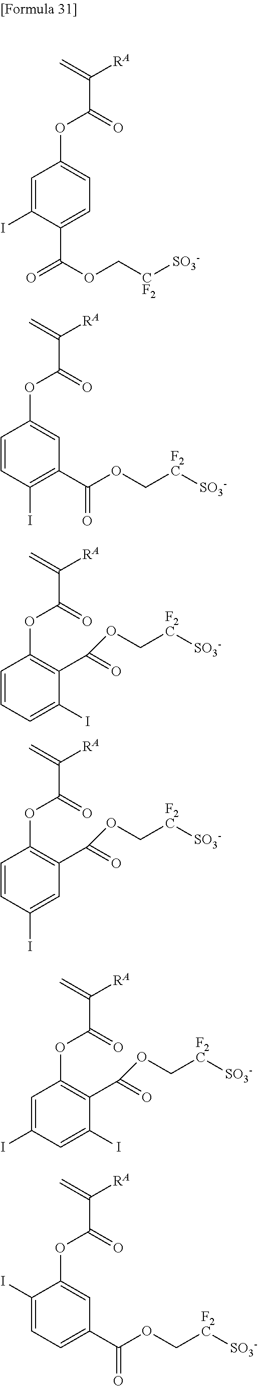

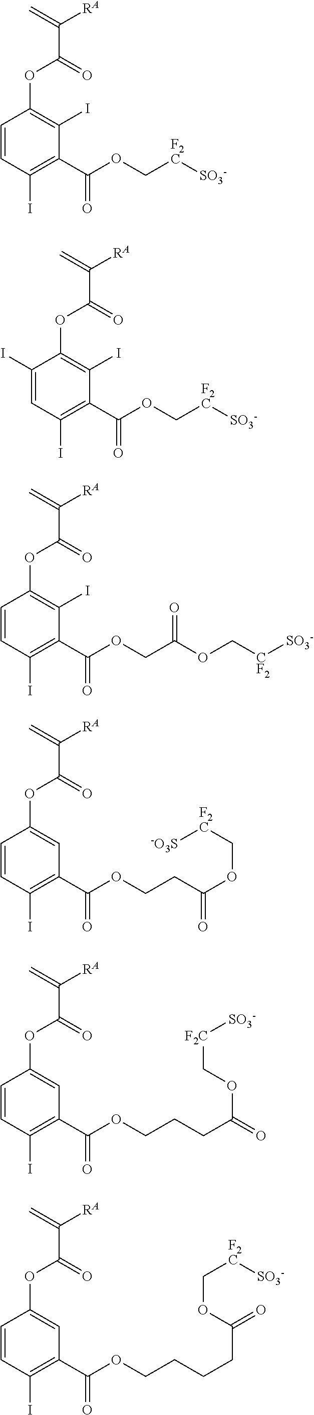

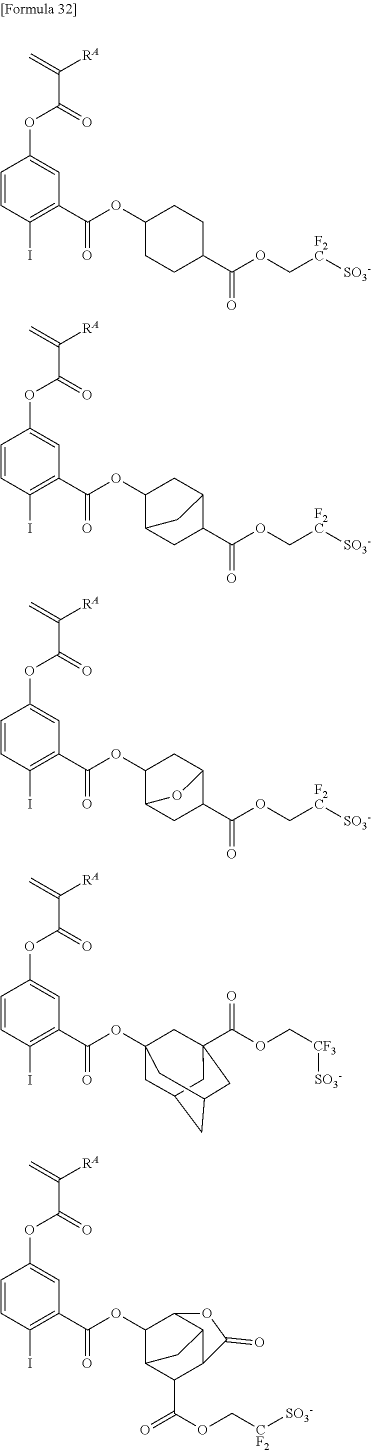

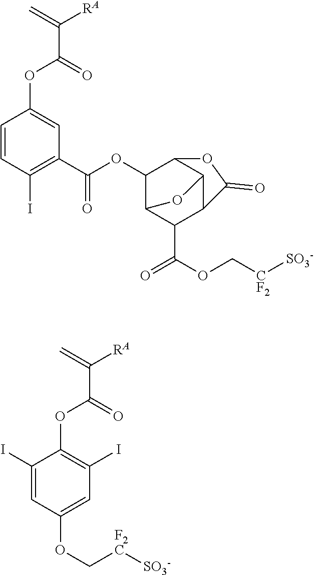

As the onium cation moiety in the monomer that affords the structural unit (V), an onium cation moiety of the acid diffusion controlling agent can be suitably employed. As the organic acid anion moiety in the structural unit (V), a carboxylic acid anion can also be applied in place of the sulfonic acid anion exemplified above. In this case, the composition may contain an acid diffusion controlling agent, or otherwise. As the monomer that affords the structural unit (V), the monomer in which the organic acid anion moiety has a polymerizable group such as a vinyl group or a (meth)acryloyl group has been exemplified above, but the onium cation moiety may be a monomer having a polymerizable group. As the monomer that affords the structural unit (IV), a monomer having a sulfonate anion in the organic acid anion moiety and a monomer having a carboxylate anion in the organic acid anion moiety may be used in combination.

When the resin contains the structural unit (V), the lower limit of the content ratio of the structural unit (V) (when a plurality of types of the structural unit (V) are contained, the total content ratio of them) is preferably 5 mol %, more preferably 10 mol %, still more preferably 12 mol % based on all the structural units composing the resin. The upper limit of the content is preferably 30 mol %, more preferably 25 mol %, still more preferably 20 mol %. When the content ratio of the structural unit (V) is adjusted to within the above range, a function of the resin as an acid generator or an acid diffusion controlling agent can be sufficiently exhibited.

The monomer that affords the structural unit (V) can be synthesized, for example, by the same method as that for a sulfonium salt having a polymerizable anion described in JP-B-5201363.

### <Another Resin>

The radiation-sensitive resin composition of the present embodiment may contain as another resin a resin having a higher mass content of fluorine atoms than that in the base resin as described above (hereinafter, also referred as “high fluorine-containing resin”). When the radiation-sensitive resin composition contains the high fluorine-containing resin, the high fluorine-containing resin can be localized in the surface layer of a resist film compared to the base resin, and as a result, the state of the surface of the resist film and the component distribution in the resist film can be controlled to a desired state.

### (Method for Synthesizing High Fluorine-Containing Resin)

The high fluorine-containing resin can be synthesized by the same method as the method for synthesizing the base resin described above.

### <Solvent>

The radiation-sensitive resin composition according to the present embodiment contains a solvent. The solvent is not particularly limited as long as the solvent is a solvent capable of dissolving or dispersing at least the onium salt, the base resin, and additives or the like contained as desired.

Examples of the solvent include an alcohol-based solvent, an ether-based solvent, a ketone-based solvent, an amide-based solvent, an ester-based solvent, and a hydrocarbon-based solvent.

Examples of the alcohol-based solvent include:
monoalcohol-based solvents having 1 to 18 carbon atoms, such as iso-propanol, 4-methyl-2-pentanol, 3-methoxybutanol, n-hexanol, 2-ethylhexanol, furfuryl alcohol, cyclohexanol, 3,3,5-trimethylcyclohexanol, and diacetone alcohol;
polyhydric alcohol-based solvents having 2 to 18 carbon atoms, such as ethylene glycol, 1,2-propylene glycol, 2-methyl-2,4-pentanediol, 2,5-hexanediol, diethylene glycol, dipropylene glycol, triethylene glycol, and tripropylene glycol; and
partially etherized polyhydric alcohol-based solvents obtained by etherifying some of hydroxy groups of the polyhydric alcohol-based solvent.

Examples of the ether-based solvent include:
dialkyl ether-based solvents, such as diethyl ether, dipropyl ether, and dibutyl ether;
cyclic ether-based solvents, such as tetrahydrofuran and tetrahydropyran;
aromatic ring-containing ether-based solvents, such as diphenyl ether and anisole (methyl phenyl ether); and
etherized polyhydric alcohol-based solvents obtained by etherifying a hydroxy group of the polyhydric alcohol-based solvent.

Examples of the ketone-based solvent include chain ketone-based solvents, such as acetone, butanone, and methyl-iso-butyl ketone;
cyclic ketone-based solvents, such as cyclopentanone, cyclohexanone, and methylcyclohexanone; and
2,4-pentanedione, acetonylacetone, and acetophenone.

Examples of the amide-based solvent include cyclic amide-based solvents, such as N,N′-dimethylimidazolidinone and N-methylpyrrolidone; and
chain amide-based solvents, such as N-methylformamide, N,N-dimethylformamide, N,N-diethylformamide, acetamide, N-methylacetamide, N,N-dimethylacetamide, and N-methylpropionamide.

Examples of the ester-based solvent include:
monocarboxylic acid ester-based solvents, such as n-butyl acetate, and ethyl lactate;
partially etherized polyhydric alcohol acetate-based solvents, such as diethylene glycol mono-n-butyl ether acetate, propylene glycol monomethyl ether acetate, and dipropylene glycol monomethyl ether acetate;
lactone-based solvents, such as γ-butyrolactone and valerolactone;
carbonate-based solvents, such as diethyl carbonate, ethylene carbonate, and propylene carbonate; and
polyvalent carboxylic acid diester-based solvents, such as propylene glycol diacetate, methoxytriglycol acetate, diethyl oxalate, ethyl acetoacetate, ethyl lactate, and diethyl phthalate.

Examples of the hydrocarbon-based solvent include:
aliphatic hydrocarbon-based solvents, such as n-hexane, cyclohexane, and methylcyclohexane; and
aromatic hydrocarbon-based solvents, such as benzene, toluene, di-iso-propylbenzene, and n-amylnaphthalene.

Among them, an ether-based solvent and an ester-based solvent are preferable, a polyhydric alcohol partial ether-based solvent, and a polyhydric alcohol partial ether acetate-based solvent are more preferable, and propylene glycol monomethyl ether and propylene glycol monomethyl ether acetate are still more preferable. The radiation-sensitive resin composition may contain one type or two or more types of solvent.

### <Other Optional Components>

The radiation-sensitive resin composition may contain, in addition to the components, other optional components. Examples of other optional components may include a cross-linking agent, a localization enhancing agent, a surfactant, an alicyclic backbone-containing compound, and a sensitizer. These other optional components may be used singly, or two or more thereof may be used in combination.

### <Method for Preparing Radiation-Sensitive Resin Composition>

The radiation-sensitive resin composition can be prepared, for example, by mixing the onium salt, the base resin, and the solvent, and if necessary, the other optional component at a prescribed ratio. The radiation-sensitive resin composition is preferably filtered through, for example, a filter having a pore diameter of about 0.05 μm to 0.4 μm after mixing. The solid matter concentration of the radiation-sensitive resin composition is usually 0.1 mass % to 50 mass %, preferably 0.5 mass % to 30 mass %, more preferably 1 mass % to 20 mass %.

### <<Pattern Formation Method>>

A pattern forming method according to the present invention includes:
(1) applying the radiation-sensitive resin composition directly or indirectly to a substrate to form a resist film (hereinafter, also referred to as a “resist film forming step”);
(2) exposing the resist film to light (hereinafter also referred to as “exposure step”); and
(3) developing the exposed resist film (hereinafter, also referred to as a “developing step”).

According to the pattern formation method, a high-quality resist pattern can be formed because of use of the radiation-sensitive resin composition superior in sensitivity and LWR performance in the exposure step. Hereinbelow, each of the steps will be described.

### [Resist Film Forming Step]

In this step (the step (1)), a resist film is formed from the radiation-sensitive resin composition. Examples of the substrate on which the resist film is formed may include those traditionally known in the art, including a silicon wafer, silicon dioxide, and a wafer coated with aluminum. An organic or inorganic antireflective film disclosed in, for example, JP-B-6-12452 or JP-A-59-93448 may be formed on the substrate. Examples of an applicating method may include a rotary coating (spin coating), flow casting, and roll coating. After the application, prebaking (PB) may be performed to volatilize the solvent in the coating film, as necessary. The temperature of PB is usually from 60 °C to 150 °C, preferably from 80 °C to 140 °C. The duration of PB is usually from 5 seconds to 600 seconds, preferably from 10 seconds to 300 seconds. The thickness of the resist film formed is preferably from 10 nm to 1,000 nm, more preferably from 10 nm to 500 nm.

When the exposure step that is the next step is performed with a radioactive-ray having a wavelength of 50 nm or less, it is preferable to use, as the base resin in the composition, a resin containing the structural units (I) and (II), and the structural unit (III) as necessary.

### [Exposure Step]

In this step (the step (2)), the resist film formed in the resist film forming step as the step (1) is exposed by irradiating with radiation through a photomask (in some cases, through an immersion medium such as water). Examples of the radiation to be used for the exposure may include an electromagnetic wave including visible ray, ultraviolet ray, far ultraviolet ray, extreme ultraviolet ray (EUV), X ray, and γ ray; and charged particle radiation such as an electron beam and α ray. Among them, far ultraviolet ray, an electron beam, or EUV is preferable. ArF excimer laser light (wavelength is 193 nm), KrF excimer laser light (wavelength is 248 nm), an electron beam, or EUV is more preferable. An electron beam having a wavelength of 50 nm or less, which is identified as the next generation exposing technology, or EUV is further preferable.

After the exposure, post exposure bake (PEB) is preferably performed to promote the dissociation of the acid-dissociable group of the resin by an acid generated from the radiation-sensitive acid generator through exposure to light in the exposed part of the resist film. As a result of the PEB, there is generated a difference in solubility into a developer between the exposed area and the unexposed area. The temperature of PEB is usually from 50 °C to 180 °C, preferably from 80 °C to 130 °C. The duration of PEB is usually from 5 seconds to 600 seconds, preferably from 10 seconds to 300 seconds.

### [Development Step]

In this step (the step (3)), the resist film exposed in the exposure step as the step (2) is developed. By this step, the predetermined resist pattern can be formed. After the development, the resist pattern is generally washed with a rinse solution such as water or alcohol, and then dried.

Examples of the developer used for the development may include, in the alkaline development, an alkaline aqueous solution obtained by dissolving at least one alkaline compound such as sodium hydroxide, potassium hydroxide, sodium carbonate, sodium silicate, sodium metasilicate, ammonia water, ethylamine, n-propylamine, diethylamine, di-n-propylamine, triethylamine, methyldiethylamine, ethyldimethylamine, triethanolamine, tetramethyl ammonium hydroxide (TMAH), pyrrole, piperidine, choline, 1,8-diazabicyclo-[5.4.0]-7-undecene, 1,5-diazabicyclo-[4.3.0]-5-nonene. Among them, an aqueous TMAH solution is preferable, and a 2.38% by mass aqueous TMAH solution is more preferable.

In the case of organic solvent development, examples of the solvent may include organic solvents such as hydrocarbon-based solvents, ether-based solvents, ester-based solvents, ketone-based solvents, and alcohol-based solvents, and solvents containing an organic solvent. Examples of the organic solvent may include one, two or more solvents listed as the solvent for the radiation-sensitive resin composition. Among them, ester-based solvents and ketone-based solvents are preferable. As the ester-based solvents, acetate-based solvents are preferable, and n-butyl acetate and amyl acetate are more preferable. As the ketone-based solvents, chain ketones are preferable, and 2-heptanone is more preferable. The content of the organic solvent in a developer is preferably 80% by mass or more, more preferably 90% by mass or more, still more preferably 95% by mass or more, particularly preferably 99% by mass or more. Examples of the component other than the organic solvent in the developer may include water and silicone oil.

Examples of the developing method may include a method including dipping a substrate in a tank filled with a developer for a given time (dipping method); a developing method including raising a developer on the surface of a substrate due to surface tension and leaving the raised developer for a given time (paddling method); a method including spraying a developer on the surface of a substrate (spraying method); and a method including injecting a developer on a substrate rolling at a constant rate while scanning a developer injection nozzle at a constant rate (dynamic dispensing method).

### EXAMPLES

Hereinafter, the present invention will be specifically described with reference to Examples, but the present invention is not limited to these Examples. Physical property values in the Examples were measured as follows.

### [Weight-Average Molecular Weight (Mw) and Number-Average Molecular Weight (Mn)]

Measurement was performed by gel permeation chromatography (GPC) with monodisperse polystyrene standards using GPC columns (G2000HXL×2, G3000HXL×1, and G4000HXL×1) manufactured by Tosoh Corporation, under analysis conditions including a flow rate: 1.0 mL/min, an elution solvent: tetrahydrofuran, a sample concentration: 1.0 mass %, an amount of sample injected: 100 μL, a column temperature: 40 °C, and a detector: a differential refractometer. The degree of dispersion (Mw/Mn) was calculated from the measurement results of Mw and Mn. The Mw and Mn of the resins synthesized below are shown in Table 1.

### <Synthesis of Resin [A]>

Monomers used for synthesis of the respective resins in the respective Examples and Comparative Examples are shown below.

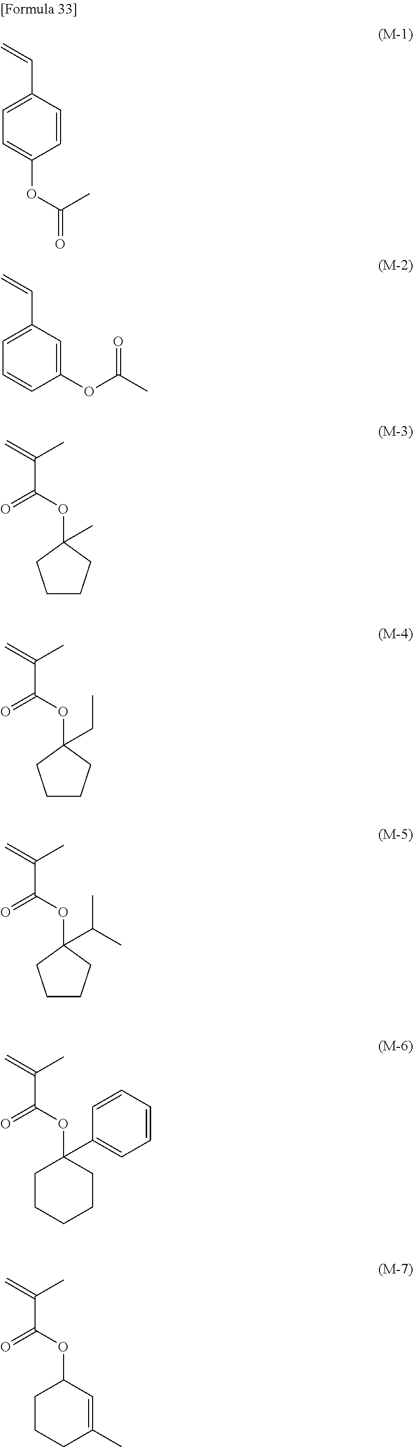

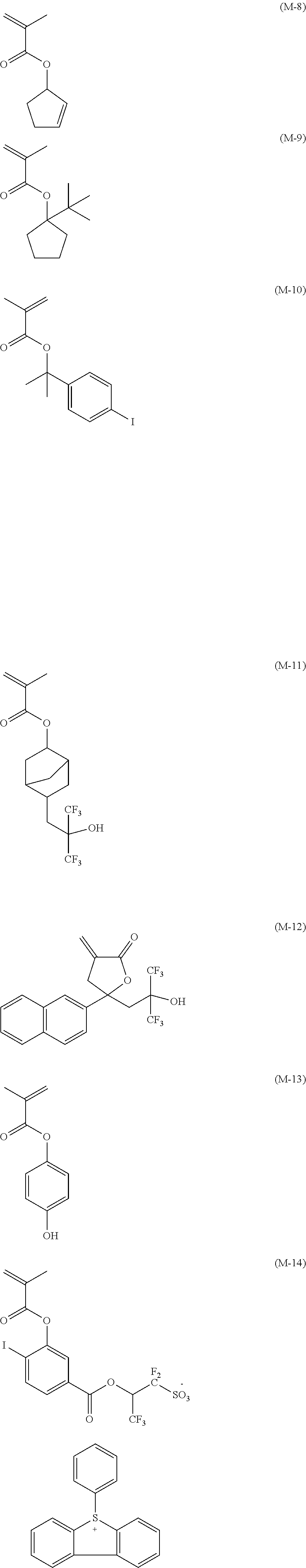

### [Resin Synthesis Example 1] Synthesis of Resin (A-1)

Compound (M-1) and compound (M-3) were dissolved in 1-methoxy-2-propanol (200 parts by mass with respect to the total amount of monomers) so as to have a molar ratio of 40/60. Next, azobisisobutyronitrile was added as an initiator in an amount of 6 mol % with respect to all the monomers to prepare a monomer solution. On the other hand, 1-methoxy-2-propanol (100 parts by mass with respect to the total amount of monomers) was added to an empty reaction vessel, followed by heating to 85 °C with stirring. Next, the monomer solution prepared above was added dropwise over 3 hours, followed by further heating at 85 °C for 3 hours. After completion of the polymerization reaction, the polymerization solution was cooled to room temperature. The cooled polymerization solution was charged into hexane (500 parts by mass with respect to the polymerization solution), and a precipitated white powder was separated by filtration. The obtained white powder was washed twice with 100 parts by mass of hexane with respect to the polymerization solution. Then, the white powder was dissolved in 1-methoxy-2-propanol (300 parts by mass). Next, methanol (500 parts by mass), triethylamine (50 parts by mass), and ultrapure water (10 parts by mass) were added, and a hydrolysis reaction was performed at 70 °C for 6 hours with stirring. After completion of the reaction, the remaining solvent was distilled off, and the obtained solid was dissolved in acetone (100 parts by mass). The obtained solution was added dropwise into 500 parts by mass of water to permit coagulation of the resin. The obtained solid was separated by filtration. The separated solid was dried at 50 °C for 12 hours to yield a white powdery resin ((A-1).

### [Resin Synthesis Examples 2 to 10] Synthesis of Resins (A-1) to (A-10)

Resins (A-2) to (A-10) were obtained in the same manner as in Resin Synthesis Example 1 except that compounds described in Table 1 were used. In Table 1, “-” indicates that the corresponding component was not used. The same applies to the following tables.

### [Resin Synthesis Example 11] Synthesis of Resin (A-11)

In a reaction vessel, 20 mmol of the compound (M-1), 40 mmol of triethylamine, 100 ml of 1-methoxy-2 propanol, and 50 ml of methanol were added, followed by heating at 70 °C for 3 hours. The solvent was distilled off to yield a hydrolysate of the compound (M-1). The hydrolysate of the compound (M-1), the compound (M-3), and the compound (M-13) were dissolved in 1-methoxy-2-propanol (200 parts by mass with respect to the total amount of monomers) such that the molar ratio was 30/60/10. Next, azobisisobutyronitrile was added as an initiator in an amount of 6 mol % with respect to all the monomers to prepare a monomer solution. On the other hand, 1-methoxy-2-propanol (100 parts by mass with respect to the total amount of monomers) was added to an empty reaction vessel, followed by heating to 85 °C with stirring. Next, the monomer solution prepared above was added dropwise over 3 hours, followed by further heating at 85 °C for 3 hours. After completion of the polymerization reaction, the polymerization solution was cooled to room temperature. The cooled polymerization solution was charged into hexane (500 parts by mass with respect to the polymerization solution), and a precipitated white powder was separated by filtration. The obtained white powder was washed twice with 100 parts by mass of hexane with respect to the polymerization solution. Thereafter, the washed white powder was dried at 50 °C for 12 hours to yield a white powdery resin (A-11).

### [Resin Synthesis Examples 12 to 13] Synthesis of Resins (A-12) to (A-13)

Resins (A-12) to (A-13) were obtained in the same manner as in Resin Synthesis Example 11 except that compounds described in Table 1 were used.

TABLE 1

| | | Monomer compound | | Monomer compound | | Monomer compound | | | |
|---|---|---|---|---|---|---|---|---|---|
| | | that affords structural | | that affords structural | | that affords structural | | | |
| | | unit (II) | | unit (I) | | unit (III) or (V) | | | |
| | | | Used | | Used | | Used | | |
| | | | amount | | amount | | amount | | |
| | Resin | Type | (mol %) | Type | (mol %) | Type | (mol %) | Mw | Mw/Mn |
| Resin Synthesis | A-1 | M-1 | 40 | M-3 | 60 | — | — | 5700 | 1.6 |
| Example 1 | | | | | | | | | |
| Resin Synthesis | A-2 | M-1 | 40 | M-4 | 60 | — | — | 5800 | 1.6 |
| Example 2 | | | | | | | | | |
| Resin Synthesis | A-3 | M-1/M-2 | 30/10 | M-5 | 60 | — | — | 6100 | 1.7 |
| Example 3 | | | | | | | | | |
| Resin Synthesis | A-4 | M-1 | 40 | M-6 | 60 | — | — | 6200 | 1.5 |
| Example 4 | | | | | | | | | |
| Resin Synthesis | A-5 | M-1 | 40 | M-7 | 60 | — | — | 5500 | 1.5 |
| Example 5 | | | | | | | | | |
| Resin Synthesis | A-6 | M-1 | 40 | M-8 | 60 | — | — | 5400 | 1.5 |
| Example 6 | | | | | | | | | |
| Resin Synthesis | A-7 | M-1 | 40 | M-9 | 60 | — | — | 6000 | 1.7 |
| Example 7 | | | | | | | | | |
| Resin Synthesis | A-8 | M-1 | 40 | M-10 | 60 | — | — | 6900 | 1.7 |
| Example 8 | | | | | | | | | |
| Resin Synthesis | A-9 | M-1 | 30 | M-3 | 60 | M-11 | 10 | 6900 | 1.7 |
| Example 9 | | | | | | | | | |
| Resin Synthesis | A-10 | M-1 | 30 | M-3 | 60 | M-12 | 10 | 6800 | 1.7 |
| Example 10 | | | | | | | | | |
| Resin Synthesis | A-11 | M-1/M- | 30/10 | M-3 | 60 | — | — | 6700 | 1.7 |
| Example 11 | | 13 | | | | | | | |
| Resin Synthesis | A-12 | M-1 | 30 | M-3 | 55 | M-14 | 15 | 6600 | 1.7 |
| Example 12 | | | | | | | | | |
| Resin Synthesis | A-13 | M-1 | 30 | M-3 | 55 | M-14/Z-13 | 12.5/2.5 | 6800 | 1.7 |
| Example 13 | | | | | | | | | |

### <Synthesis of Acid Diffusion Controlling Agent [Z]>

### Synthesis Example 1 of Acid Diffusion Controlling Agent

### (Synthesis of Acid Diffusion Controlling Agent (Z-1))

An acid diffusion controlling agent (Z-1) was synthesized according to the following reaction scheme.

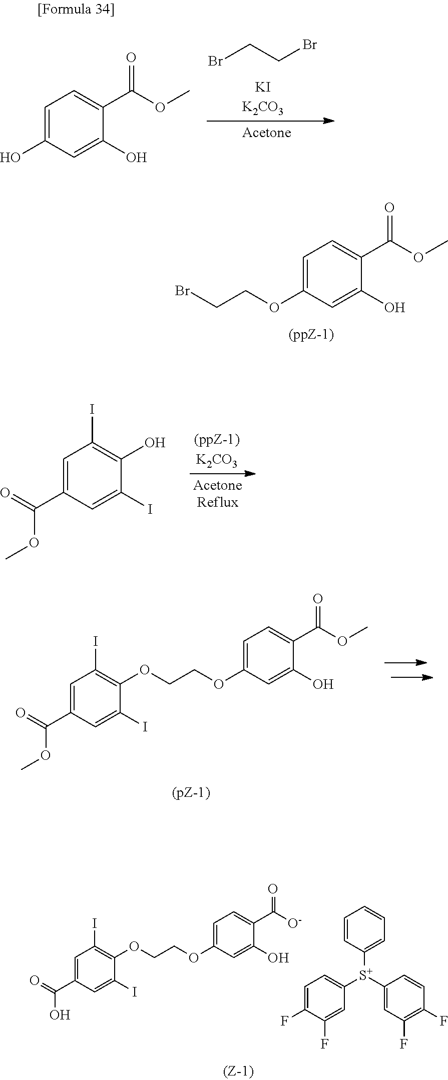

To a reaction vessel, 29.74 mmol of methyl 2,4-dihydroxybenzoate, 148.7 mmol of potassium carbonate, and 100 ml of acetone were added. The mixture was heated and stirred at 55 °C for 2 hours, and then cooled to room temperature. After 8.92 mmol of potassium iodide was added, 59.47 mmol of 1,2-dibromoethane was added dropwise, followed by heating and stirring again at 55 °C for 5 hours. After cooling to room temperature, the precipitated solid was filtered, and the filtrate was concentrated. Purification was performed by silica gel column chromatography to yield (ppZ-1).

To a reaction vessel, 20 mmol of methyl 4-hydroxy-3,5-diiodobenzoate, 20 mmol of potassium carbonate, 20 mmol of (ppZ-1), and 100 ml of acetone were added, followed by heating and stirring at 55 °C for 5 hours. After cooling to room temperature, the precipitated solid was filtered, and the filtrate was concentrated. Purification was performed by silica gel column chromatography to yield (pZ-1).

To a reaction vessel, 12 mmol of (pZ-1), 36 mmol of lithium hydroxide, 120 ml of tetrahydrofuran, and 20 ml of ultrapure water were added, followed by stirring at room temperature for 8 hours. The reaction solution was concentrated, followed by addition of 50 ml of ultrapure water. Then, the mixture was extracted twice with 100 ml of dichloromethane. The aqueous layer was neutralized with 2 M hydrochloric acid, and the precipitated solid was filtered and washed with ultrapure water. To a reaction vessel, the obtained solid, 12 mmol of sodium bicarbonate, and 50 ml of ultrapure water were added, followed by stirring for 30 minutes. Next, 12 mmol of bis(3,4-difluorophenyl)phenylsulfonium chloride and 50 ml of dichloromethane were added, followed by vigorous stirring for 3 hours. The organic layer was separated and washed twice with 50 ml of ultrapure water. The organic layer was concentrated to yield (Z-1).

### [Synthesis Examples 2 to 5 and 11 to 13 of Acid Diffusion Controlling Agent] (Synthesis of Acid Diffusion Controlling Agents (Z-2) to (Z-5) and (Z-11) to (Z-13))

Acid diffusion controlling agents (Z-2) to (Z-5) and (Z-11) to (Z-13) were synthesized in the same manner as in Synthesis Example 1 of acid diffusion controlling agent except that the used substrate was appropriately changed in Synthesis Example 1 of acid diffusion controlling agent.

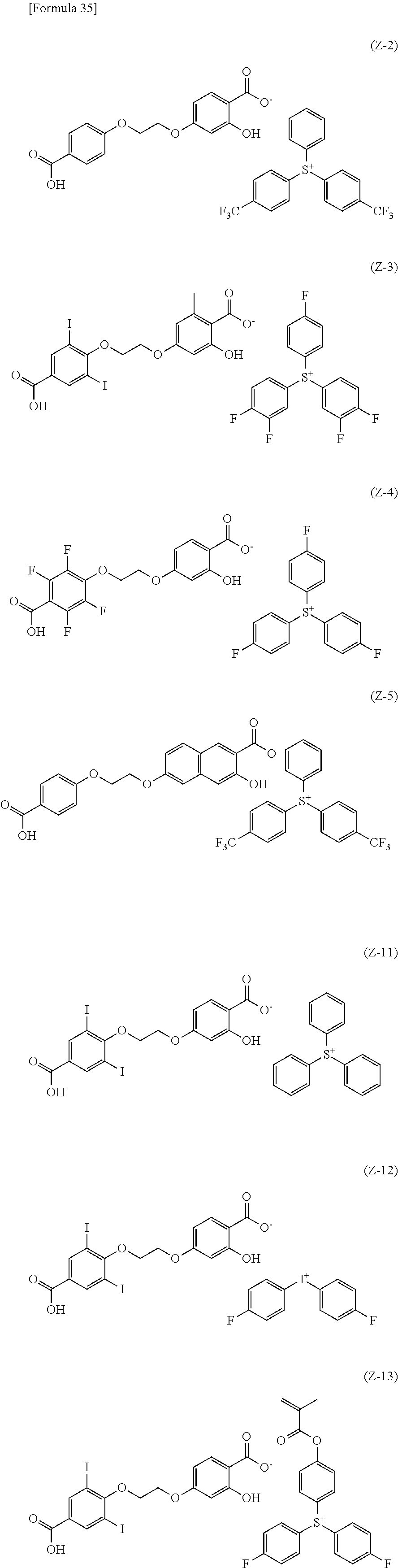

### Synthesis Example 6 of Acid Diffusion Controlling Agent

### (Synthesis of Acid Diffusion Controlling Agent (Z-6))

An acid diffusion controlling agent (Z-6) was synthesized according to the following reaction scheme.

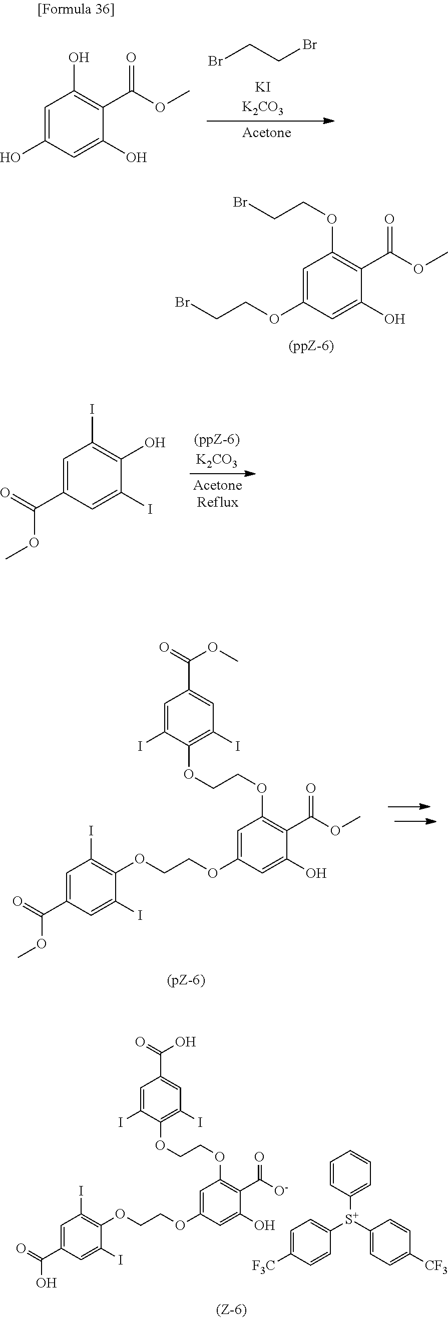

To a reaction vessel, 30 mmol of methyl 2,4,6-trihydroxybenzoate, 230 mmol of potassium carbonate, and 150 ml of acetone were added. The mixture was heated and stirred at 55 °C for 2 hours, and then cooled to room temperature. After 18 mmol of potassium iodide was added, 120 mmol of 1,2-dibromoethane was added dropwise, followed by heating and stirring again at 55 °C for 5 hours. After cooling to room temperature, the precipitated solid was filtered, and the filtrate was concentrated. Purification was performed by silica gel column chromatography to yield (ppZ-6).

To a reaction vessel, 20 mmol of methyl 4-hydroxy-3,5-diiodobenzoate, 20 mmol of potassium carbonate, 10 mmol of (ppZ-6), and 100 ml of acetone were added, followed by heating and stirring at 55 °C for 5 hours. After cooling to room temperature, the precipitated solid was filtered, and the filtrate was concentrated. Purification was performed by silica gel column chromatography to yield (pZ-6).

To a reaction vessel, 8 mmol of (pZ-6), 40 mmol of lithium hydroxide, 60 ml of tetrahydrofuran, and 10 ml of ultrapure water were added, followed by stirring at room temperature for 8 hours. The reaction solution was concentrated, followed by addition of 50 ml of ultrapure water. Then, the mixture was extracted twice with 100 ml of dichloromethane. The aqueous layer was neutralized with 2 M hydrochloric acid, and the precipitated solid was filtered and washed with ultrapure water. To a reaction vessel, the obtained solid, 8 mmol of sodium bicarbonate, and 50 ml of ultrapure water were added, followed by stirring for 30 minutes. Next, 8 mmol of bis(4-trifluoromethylphenyl)phenylsulfonium chloride and 50 ml of dichloromethane were added, followed by vigorous stirring for 3 hours. The organic layer was separated and washed twice with 50 ml of ultrapure water. The organic layer was concentrated to yield (Z-6).

### Synthesis Example 7 of Acid Diffusion Controlling Agent

### (Synthesis of Acid Diffusion Controlling Agent (Z-7))

An acid diffusion controlling agent (Z-7) was synthesized according to the following reaction scheme.

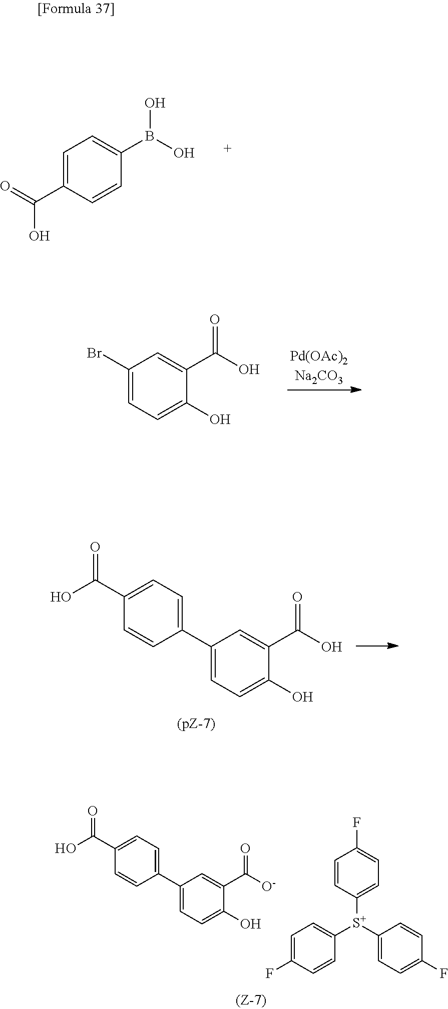 [SMILES: O=C(O)c1ccc(B(O)O)cc1 ; O=C(O)c1cc(Br)ccc1O ; O=C(O)c1ccc(-c2ccc(O)c(C(=O)O)c2)cc1 ; O=C(O)c1ccc(-c2ccc(O)c(C(=O)[O-])c2)cc1 ; Fc1ccc([S+](c2ccc(F)cc2)c2ccc(F)cc2)cc1]

To a reaction vessel, 20 mmol of 5-bromosalicylic acid, 22 mmol of 4-carboxyphenylboronic acid, 0.2 mmol of palladium acetate, 60 mmol of sodium carbonate, and 80 ml of ultrapure water were added, followed by stirring at room temperature for 1 hour. Then, 320 ml of ultrapure water was added, followed by filtration. The filtrate was neutralized with 2 M hydrochloric acid. The precipitated solid was filtered and dried, and then dissolved in 300 ml of acetone, followed by filtration again. To the filtrate, 500 ml of ultrapure water was added, followed by recrystallized to yield (pZ-7).

To a reaction vessel, 10 mmol of (pZ-7), 10 mmol of sodium bicarbonate, and 50 ml of ultrapure water were added, followed by stirring for 30 minutes. Next, 10 mmol of tris(4-fluorophenyl)sulfonium bromide and 50 ml of dichloromethane were added, followed by vigorous stirring for 3 hours. The organic layer was separated and washed twice with 50 ml of ultrapure water. The organic layer was concentrated to yield (Z-7).

### Synthesis Example 8 of Acid Diffusion Controlling Agent

### (Synthesis of Acid Diffusion Controlling Agent (Z-8))

An acid diffusion controlling agent (Z-8) was synthesized according to the following reaction scheme.

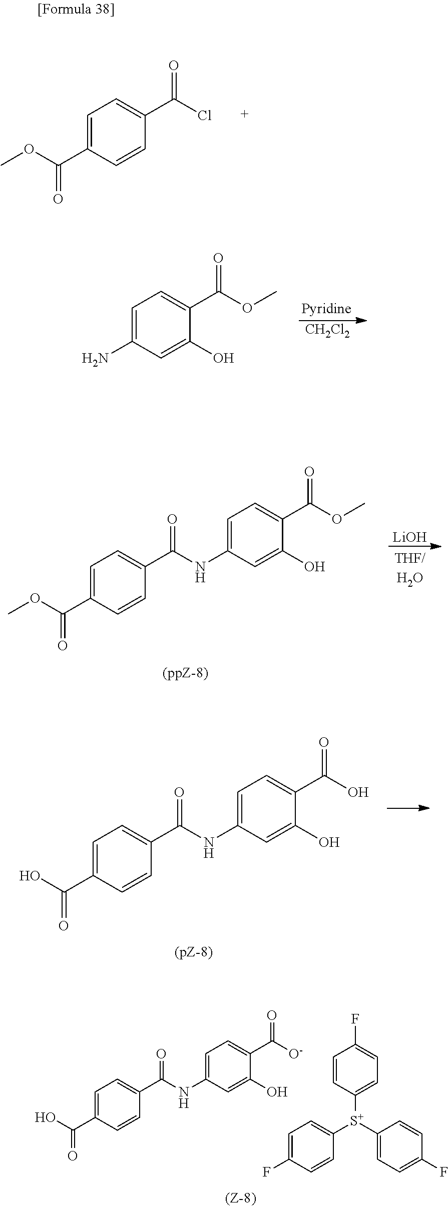

To a reaction vessel, 30 mmol of methyl 4-aminosalicylate and 100 ml of dichloromethane were added, followed by cooling to 0 °C. Next, 33 mmol of methyl 4-chloroformylbenzoate was added slowly, followed by dropwise addition of 45 mmol of pyridine. After completion of the dropwise addition, the temperature was returned to room temperature, followed by stirring for 5 hours. After completion of the reaction, 50 ml of ultrapure water was added to quench the reaction, and the organic layer was washed twice with 50 ml of a saturated aqueous sodium bicarbonate solution, twice with 50 ml of a saturated aqueous ammonium chloride solution, and once with 50 ml of a saturated saline solution. The organic layer was dried over sodium sulfate, concentrated, and purified by silica gel column chromatography to yield (ppZ-8).

To a reaction vessel, 15 mmol of (ppZ-8), 45 mmol of lithium hydroxide, 170 ml of tetrahydrofuran, and 30 ml of ultrapure water were added, followed by stirring at room temperature for 5 hours. The reaction solution was concentrated, followed by addition of 140 ml of ultrapure water. Then, the mixture was neutralized with 2 M hydrochloric acid. The precipitated solid was filtered and washed with ultrapure water to yield (pZ-8). To a reaction vessel, 10 mmol of the obtained (pZ-8), 15 mmol of sodium bicarbonate, and 50 ml of ultrapure water were added, followed by stirring for 30 minutes. Next, 15 mmol of tris(4-fluorophenyl)sulfonium bromide and 100 ml of dichloromethane were added, followed by vigorous stirring for 3 hours. The organic layer was separated and washed twice with 50 ml of ultrapure water. The organic layer was concentrated to yield (Z-8).

### Synthesis Example 9 of Acid Diffusion Controlling Agent

### (Synthesis of Acid Diffusion Controlling Agent (Z-9))

An acid diffusion controlling agent (Z-9) was synthesized according to the following reaction scheme.

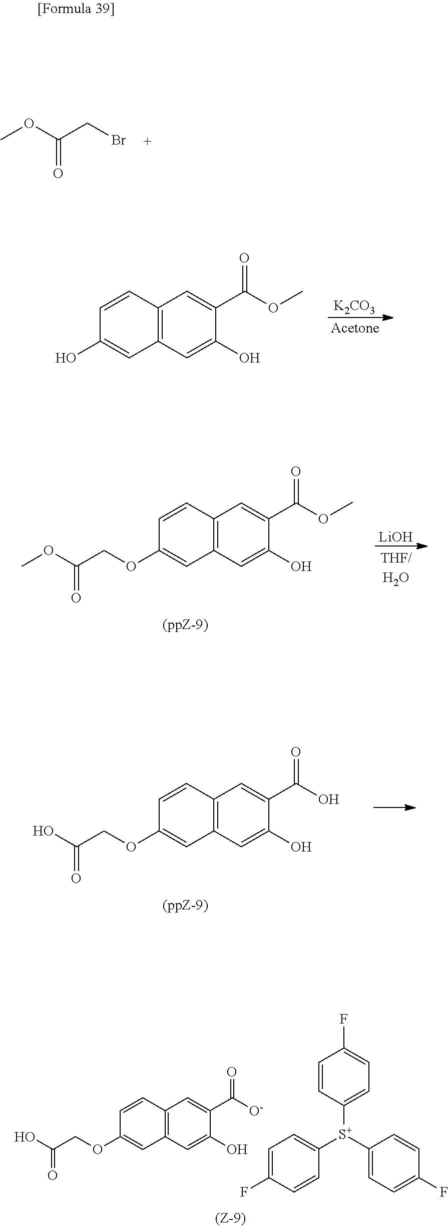

To a reaction vessel, 20 mmol of methyl 3,7-dihydroxynaphthoate, 40 mmol of potassium carbonate, and 50 ml of acetone were added, followed by stirring at 55 °C for 1 hour. The mixture was cooled to 0 °C, followed by dropwise addition of 22 mmol of methyl bromoacetate. Then, the mixture was stirred at 55 °C for 3 hours. After cooling to room temperature, the solid was filtered, and the filtrate was concentrated. Purification was performed by silica gel column chromatography to yield (ppZ-9).

To a reaction vessel, 15 mmol of (ppZ-9), 45 mmol of lithium hydroxide, 170 ml of tetrahydrofuran, and 30 ml of ultrapure water were added, followed by stirring at room temperature for 5 hours. The reaction solution was concentrated, followed by addition of 140 ml of ultrapure water. Then, the mixture was neutralized with 2 M hydrochloric acid. The precipitated solid was filtered and washed with ultrapure water to yield (pZ-9). To a reaction vessel, 10 mmol of the obtained (pZ-9), 15 mmol of sodium bicarbonate, and 50 ml of ultrapure water were added, followed by stirring for 30 minutes. Next, 15 mmol of bis(3,4-difluorophenyl) (4-fluorophenyl)sulfonium chloride and 100 ml of dichloromethane were added, followed by vigorous stirring for 3 hours. The organic layer was separated and washed twice with 50 ml of ultrapure water. The organic layer was concentrated to yield (Z-9).

### Synthesis Example 10 of Acid Diffusion Controlling Agent

### (Synthesis of Acid Diffusion Controlling Agent (Z-10))

An acid diffusion controlling agent (Z-10) was synthesized according to the following reaction scheme.

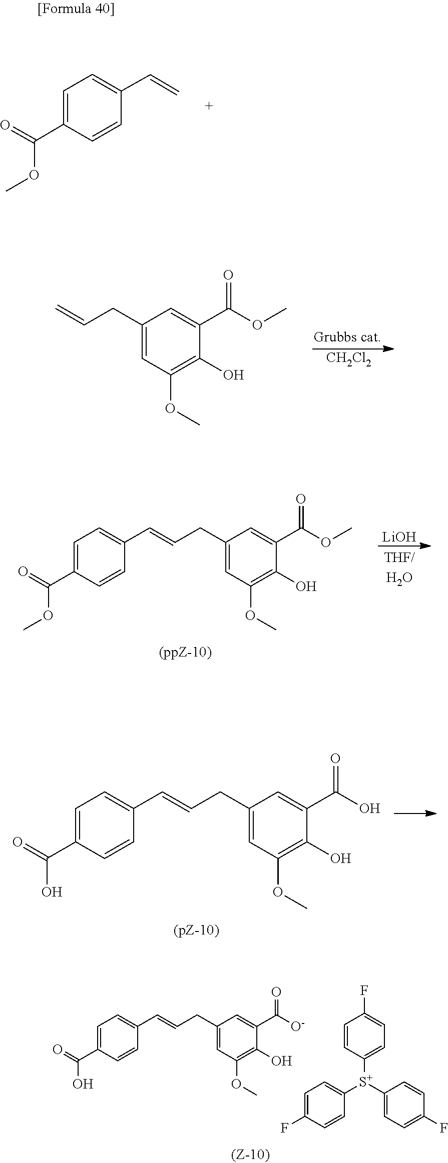

To a reaction vessel, 20 mmol of methyl 4-vinylbenzoate, 40 mmol of methyl 5-allyl-3 methoxysalicylate, 0.2 mmol of Grubbs catalyst 2nd generation (1,3-bis(2,4,6-trimethylphenyl)-2-imidazolidinylidene)dichloro(phenylmethylene) (tricyclohexyl phosphine)ruthenium, and 60 ml of dichloromethane were added, followed by stirring for 18 hours. The reaction solution was concentrated and purified by silica gel column chromatography to yield (ppZ-10).

To a reaction vessel, 10 mmol of (ppZ-10), 30 mmol of lithium hydroxide, 100 ml of tetrahydrofuran, and 20 ml of ultrapure water were added, followed by stirring at room temperature for 5 hours. The reaction solution was concentrated, followed by addition of 100 ml of ultrapure water. Then, the mixture was neutralized with 2 M hydrochloric acid. The precipitated solid was filtered, washed with ultrapure water, and dried to yield (pZ-10). To a reaction vessel, 8 mmol of (pZ-10), 8 mmol of sodium bicarbonate, and 50 ml of ultrapure water were added, followed by stirring for 30 minutes. Next, 8 mmol of tris(4-fluorophenyl)sulfonium bromide and 50 ml of dichloromethane were added, followed by vigorous stirring for 3 hours. The organic layer was separated and washed twice with 50 ml of ultrapure water. The organic layer was concentrated to yield (Z-10).

### <Method for Preparing Radioactive-Ray-Sensitive Composition>

Radiation-sensitive acid generators [B], acid diffusion controlling agents [C], and solvents [D] used for the preparation of the radiation-sensitive resin compositions of the following Examples and Comparative Examples are shown below.

### <[B] Radiation-Sensitive Acid Generator>

Compounds represented by the following formulas (B-1) to (B-9) were used as the radiation-sensitive acid generator [B].

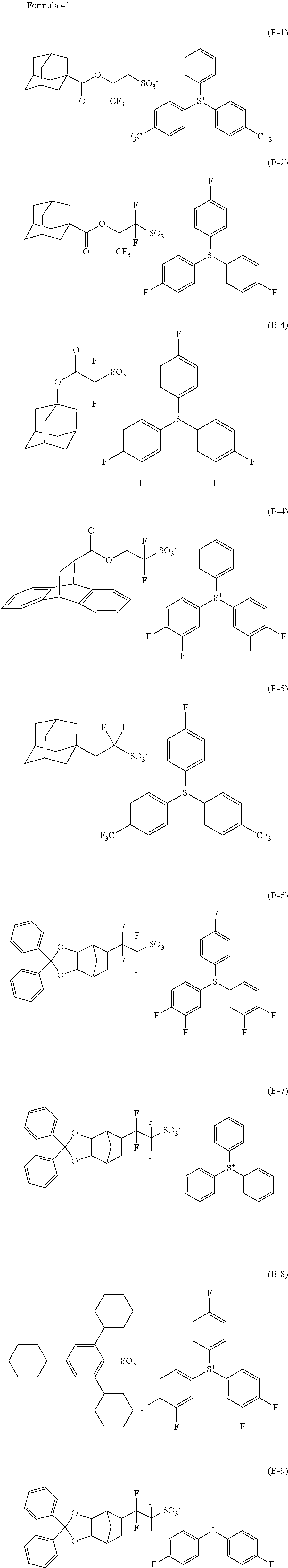

### <Acid Diffusion Controlling Agent [C]>

As the acid diffusion controlling agent [C], acid diffusion controlling agents (Z-1) to (Z-13) and compounds represented by the following formulas (C-1) to (C-4) were used.

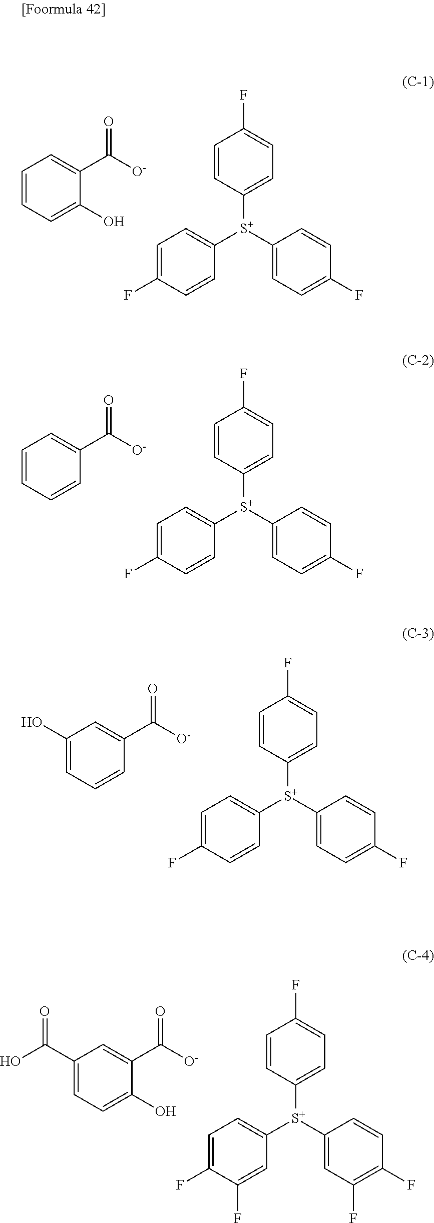

### Solvent [D]

D-1: Propylene glycol monomethyl ether acetate
D-2: Propylene glycol 1-monomethyl ether

### Example 1

A radiation-sensitive resin composition (R-1) was prepared by blending and mixing 100 parts by mass of the resin (A-1) [A], 20 parts by mass of (B-1) as the radiation-sensitive acid generator [B], 20 mol % of (Z-1) as the acid diffusion inhibitor [C] with respect to (B-1), 4,800 parts by mass of (D-1) as the solvent [D], and 2,000 parts by mass of (D-2), and then passing the mixture through a 0.20 μm filter.

### Examples 2 to 33 and Comparative Examples 1 to 5

Radiation-sensitive resin compositions (R-2) to (R-33) and (CR-1) to (CR-5) were prepared in the same manner as in Example 1 except that the types and the blending amounts of respective components shown in the following Table 2 were used. For (CR-4) in Comparative Example 4, the solubility of (C-4) in a solvent was so low that a uniform radiation-sensitive resin composition was not obtained, and accordingly, various evaluations described below were not performed, which were indicated as “-” in Table 3.

TABLE 2

| | | | | | | Acid diffusion | | | |
|---|---|---|---|---|---|---|---|---|---|
| | | | | Radiation- | | inhibitor [C] | | | |
| | Radiation- | | | sensitive acid | | | mol % with | | |
| | sensitive | Resin [A] | | generator [B] | | | respect to | | |
| | resin | | Parts by | | Parts by | | component | Solvent | Parts by |
| | composition | Type | mass | Type | mass | Type | [B] | [D] | mass |
| Example 1 | R-1 | A-1 | 100 | B-1 | 20 | Z-1 | 20 | D-1/D-2 | 4800/2000 |
| Example 2 | R-2 | A-1 | 100 | B-1 | 20 | Z-2 | 20 | D-1/D-2 | 4800/2000 |
| Example 3 | R-3 | A-1 | 100 | B-1 | 20 | Z-3 | 20 | D-1/D-2 | 4800/2000 |
| Example 4 | R-4 | A-1 | 100 | B-1 | 20 | Z-4 | 20 | D-1/D-2 | 4800/2000 |
| Example 5 | R-5 | A-1 | 100 | B-1 | 20 | Z-5 | 20 | D-1/D-2 | 4800/2000 |
| Example 6 | R-6 | A-1 | 100 | B-1 | 20 | Z-6 | 20 | D-1/D-2 | 4800/2000 |
| Example 7 | R-7 | A-1 | 100 | B-1 | 20 | Z-7 | 20 | D-1/D-2 | 4800/2000 |
| Example 8 | R-8 | A-1 | 100 | B-1 | 20 | Z-8 | 20 | D-1/D-2 | 4800/2000 |
| Example 9 | R-9 | A-1 | 100 | B-1 | 20 | Z-9 | 20 | D-1/D-2 | 4800/2000 |
| Example 10 | R-10 | A-1 | 100 | B-1 | 20 | Z-10 | 20 | D-1/D-2 | 4800/2000 |
| Example 11 | R-11 | A-1 | 100 | B-1 | 20 | Z-11 | 20 | D-1/D-2 | 4800/2000 |
| Example 12 | R-12 | A-1 | 100 | B-1 | 20 | Z-12 | 20 | D-1/D-2 | 4800/2000 |
| Example 13 | R-13 | A-1 | 100 | B-2 | 20 | Z-1 | 20 | D-1/D-2 | 4800/2000 |
| Example 14 | R-14 | A-1 | 100 | B-3 | 20 | Z-1 | 20 | D-1/D-2 | 4800/2000 |
| Example 15 | R-15 | A-1 | 100 | B-4 | 20 | Z-1 | 20 | D-1/D-2 | 4800/2000 |
| Example 16 | R-16 | A-1 | 100 | B-5 | 20 | Z-1 | 20 | D-1/D-2 | 4800/2000 |
| Example 17 | R-17 | A-1 | 100 | B-6 | 20 | Z-1 | 20 | D-1/D-2 | 4800/2000 |
| Example 18 | R-18 | A-1 | 100 | B-7 | 20 | Z-1 | 20 | D-1/D-2 | 4800/2000 |
| Example 19 | R-19 | A-1 | 100 | B-8 | 20 | Z-1 | 20 | D-1/D-2 | 4800/2000 |
| Example 20 | R-20 | A-1 | 100 | B-9 | 20 | Z-1 | 20 | D-1/D-2 | 4800/2000 |
| Example 21 | R-21 | A-1 | 100 | B-1/B-4 | 10/10 | Z-1 | 20 | D-1/D-2 | 4800/2000 |
| Example 22 | R-22 | A-2 | 100 | B-1 | 20 | Z-1 | 20 | D-1/D-2 | 4800/2000 |
| Example 23 | R-23 | A-3 | 100 | B-1 | 20 | Z-1 | 20 | D-1/D-2 | 4800/2000 |
| Example 24 | R-24 | A-4 | 100 | B-1 | 20 | Z-1 | 20 | D-1/D-2 | 4800/2000 |
| Example 25 | R-25 | A-5 | 100 | B-1 | 20 | Z-1 | 20 | D-1/D-2 | 4800/2000 |
| Example 26 | R-26 | A-6 | 100 | B-1 | 20 | Z-1 | 20 | D-1/D-2 | 4800/2000 |
| Example 27 | R-27 | A-7 | 100 | B-1 | 20 | Z-1 | 20 | D-1/D-2 | 4800/2000 |
| Example 28 | R-28 | A-8 | 100 | B-1 | 20 | Z-1 | 20 | D-1/D-2 | 4800/2000 |
| Example 29 | R-29 | A-9 | 100 | B-1 | 20 | Z-1 | 20 | D-1/D-2 | 4800/2000 |
| Example 30 | R-30 | A-10 | 100 | B-1 | 20 | Z-1 | 20 | D-1/D-2 | 4800/2000 |
| Example 31 | R-31 | A-11 | 100 | B-1 | 20 | Z-1 | 20 | D-1/D-2 | 4800/2000 |
| Example 32 | R-32 | A-12 | 100 | — | — | Z-1 | 20 | D-1/D-2 | 2000/4800 |
| Example 33 | R-33 | A-13 | — | — | — | — | — | D-1/D-2 | 2000/4800 |
| Comparative | CR-1 | A-1 | 100 | B-1 | 20 | C-1 | 20 | D-1/D-2 | 4800/2000 |
| Example 1 | | | | | | | | | |
| Comparative | CR-2 | A-1 | 100 | B-1 | 20 | C-2 | 20 | D-1/D-2 | 4800/2000 |
| Example 2 | | | | | | | | | |
| Comparative | CR-3 | A-1 | 100 | B-1 | 20 | C-3 | 20 | D-1/D-2 | 4800/2000 |
| Example 3 | | | | | | | | | |
| Comparative | CR-4 | A-1 | 100 | B-1 | 20 | C-4 | 20 | D-1/D-2 | 4800/2000 |
| Example 4 | | | | | | | | | |
| Comparative | CR-5 | A-1 | 100 | B-7 | 20 | Z-11 | 20 | D-1/D-2 | 4800/2000 |
| Example 5 | | | | | | | | | |

### <Formation of Resist Pattern> (EUV Exposure, Alkaline Development)

Each of the radiation-sensitive resin compositions prepared above was applied using a spin coater (CLEAN TRACK ACT12 manufactured by Tokyo Electron Ltd.) onto the surface of a 12-inch silicon wafer on which a lower layer film (AL412 (manufactured by Brewer Science)) having a thickness of 50 nm had been formed. After PB was performed at 130 °C for 60 seconds, cooling was performed at 23 °C for 30 seconds to form a resist film having a thickness of 50 nm. Then, the resist film was irradiated with EUV light using an EUV scanner (type “NXE3300”, manufactured by ASML, NA=0.33, lighting condition: Conventional, s=0.89, Mask imecDEFECT32FFR02). The resist film was subjected to PEB at 110 °C for 60 seconds. Then, development was performed at 23 °C for 30 seconds using a 2.38 wt % aqueous TMAH solution to form a positive 32 nm line-and-space pattern.

### <Formation of Resist Pattern> (KrF Exposure, Alkali Development)

Each of the radiation-sensitive resin compositions prepared as above was applied using a spin coater (CLEAN TRACK ACT12 manufactured by Tokyo Electron Ltd.) onto the surface of a 12-inch silicon wafer having a lower layer film with a film thickness of 20 nm (DUV42 (manufactured by Nissan Chemical Corporation)). After PB was performed at 130 °C for 60 seconds, cooling was performed at 23 °C for 30 seconds to form a resist film having a film thickness of 50 nm. Next, this resist film was irradiated with KrF light using a KrF exposure machine (Model: “S210D”, manufactured by Nikon Corporation, NA=0.55, illumination conditions: Annular s=0.8, mask 150 nmLS). The resist film was subjected to PEB at 110 °C for 60 seconds. Then, development was performed at 23 °C for 30 seconds using a 2.38 wt % aqueous TMAH solution to form a positive 150 nm line-and-space pattern.

### <Evaluation>

The sensitivity and LWR performance of each of the radiation-sensitive resin compositions were evaluated by measuring each of the formed resist patterns according to the following method. A scanning electron microscope (“CG-4100” manufactured by Hitachi High-Technologies Corporation) was used for measuring the length of the resist pattern. The evaluation results are shown in the following Table 3.

### [Sensitivity]

An exposure amount at which a 32 nm line-and-space pattern was formed in the formation of the EUV resist pattern was defined as an optimum exposure amount, and the optimum exposure amount was defined as sensitivity (mJ/cm²). When the optimum exposure dose was 30 mJ/cm² or less, the sensitivity was determined as “good”, and when the optimum exposure dose exceeded 30 mJ/cm², the sensitivity was determined as “poor”.

### [LWR Performance]

Using the scanning electron microscope, the resist pattern formed by the EUV exposure was observed from above. Line widths were measured at a total of 50 optional points. A 3 sigma value was obtained from the distribution of the measurement values, and defined as LWR (unit: nm). A smaller value of LWR indicates smaller wobble of the line and better LWR performance. The smaller the LWR performance, the better. The LWR performance was determined as “good” when the value was 3.5 nm or less, and “poor” when the value exceeded 3.5 nm.

### [Storage Stability]

After preparation of the radiation-sensitive resin composition, the radiation-sensitive resin composition was stored at −15 °C for 2 weeks or at 35 °C for 2 weeks. Thereafter, in the resist pattern formation by KrF exposure described above, an exposure amount at which a 150 nm line-and-space pattern was formed was determined, and the amount was defined as the sensitivity. On the basis of the sensitivity S_{H} of the radiation-sensitive resin composition stored at −15 °C for 2 weeks, the rate in change in sensitivity S_{H} of the radiation-sensitive resin composition stored at 35 °C for 2 weeks was determined based on the following formula. When the absolute value of the rate in change was more than 1.0%, the storage stability was determined as “poor”. Otherwise, the storage stability was determined as “good”. The rate in change is represented by a negative value when the sensitivity is increased, and is represented by a positive value when the sensitivity is decreased. Accordingly, when the rate in change is −1.0% or more and 1.0% or less, the storage stability can be determined as “good”.

Rate in change (%)={(S_{H}−S_{L})/S_{L}}×100

TABLE 3

| | Radiation- | Optimum exposure | | Storage |
|---|---|---|---|---|
| | sensitive resin | amount | LWR | stability |
| | composition | (mJ/cm²) | (nm) | (%) |
| Example 1 | R-1 | 27 | 3.0 | 0.4 |
| Example 2 | R-2 | 29 | 3.3 | 0.5 |
| Example 3 | R-3 | 25 | 3.0 | −0.1 |
| Example 4 | R-4 | 28 | 3.3 | 0 |
| Example 5 | R-5 | 29 | 3.4 | −0.5 |
| Example 6 | R-6 | 28 | 2.9 | 0 |
| Example 7 | R-7 | 29 | 3.3 | 0.4 |
| Example 8 | R-8 | 25 | 3.4 | 0.3 |
| Example 9 | R-9 | 29 | 3.3 | −0.4 |
| Example 10 | R-10 | 27 | 3.4 | −0.2 |
| Example 11 | R-11 | 29 | 2.9 | 0 |
| Example 12 | R-12 | 27 | 2.9 | 0.3 |
| Example 13 | R-13 | 27 | 3.3 | −0.1 |
| Example 14 | R-14 | 25 | 3.3 | 0 |
| Example 15 | R-15 | 25 | 3.4 | 0 |
| Example 16 | R-16 | 28 | 3.3 | −0.1 |
| Example 17 | R-17 | 25 | 3.3 | 0 |
| Example 18 | R-18 | 29 | 3.5 | 0.3 |
| Example 19 | R-19 | 29 | 3.2 | 0.3 |
| Example 20 | R-20 | 28 | 3.0 | 0.4 |
| Example 21 | R-21 | 28 | 3.4 | −0.2 |
| Example 22 | R-22 | 29 | 3.5 | −0.4 |
| Example 23 | R-23 | 27 | 3.0 | −0.2 |
| Example 24 | R-24 | 29 | 3.4 | −0.3 |
| Example 25 | R-25 | 26 | 3.4 | 0.5 |
| Example 26 | R-26 | 26 | 3.0 | −0.3 |
| Example 27 | R-27 | 28 | 3.0 | −0.1 |
| Example 28 | R-28 | 29 | 3.4 | 0.1 |
| Example 29 | R-29 | 25 | 3.1 | −0.1 |
| Example 30 | R-30 | 29 | 3.1 | 0 |
| Example 31 | R-31 | 29 | 3.3 | 0.1 |
| Example 32 | R-32 | 28 | 3.4 | 0.1 |
| Example 33 | R-33 | 26 | 2.9 | 0.2 |
| Comparative | CR-1 | 31 | 4.1 | −1.2 |
| Example 1 | | | | |
| Comparative | CR-2 | 33 | 4.3 | −1.1 |
| Example 2 | | | | |
| Comparative | CR-3 | 30 | 4.1 | −1.3 |
| Example 3 | | | | |
| Comparative | CR-4 | — | — | — |
| Example 4 | | | | |
| Comparative | CR-5 | 35 | 3.7 | 0.1 |
| Example 5 | | | | |

As is apparent from the results in Table 3, the radiation-sensitive resin compositions of Examples were better in sensitivity, LWR, and storage stability than the radiation-sensitive resin compositions of Comparative Examples.

### INDUSTRIAL APPLICABILITY

According to the radiation-sensitive resin composition and the resist pattern formation method of the present invention, the sensitivity and LWR of the radiation-sensitive resin composition can be improved as compared with the conventional one while the storage stability is maintained. Therefore, the radiation-sensitive composition and the pattern forming method can be suitably used for the formation of a fine resist pattern in a lithography process for various electronic devices such as semiconductor devices and liquid crystal devices.

1: A radiation-sensitive resin composition, comprising:
one or more onium salts each comprising an organic acid anion moiety and an onium cation moiety;
a resin comprising a structural unit comprising an acid-dissociable group; and
a solvent,
wherein an onium cation moiety of at least a part of the one or more onium salts is a fluorine-containing onium cation moiety comprising a fluorine atom, and
an organic acid anion moiety of at least a part of the one or more onium salts is an organic acid anion moiety (1) represented by formula (1): 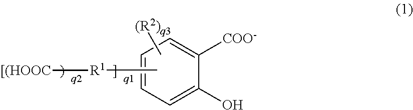
wherein in the formula (1),
q1 is an integer of 1 to 4, when q1 is 2 or more, a plurality of R¹s are identical to or different from each other,
when q1 is 1, q2 is an integer of 1 to 4, when q1 is 2 or more, a plurality of q2s are each independently an integer of 0 to 4, provided that at least one q2 is 1 or more,
q3 is an integer of 0 to 3, when q3 is 2 or more, a plurality of R²s are identical to or different from each other,
q1 and q3 satisfy q1+q3≤4,
R¹ is a (q2+1)-valent organic group, and when q1 is two or more, two R¹s optionally taken together represent a ring structure having 5 to 20 ring numbers together with the carbon atoms of the benzene ring in the formula (1) to which the two R¹s are bonded, and
R² is a halogen atom, a hydroxy group, an alkyl group, an alkoxy group, a cyano group, a nitro group, or an amino group.

2: The radiation-sensitive resin composition according to claim 1, wherein the one or more onium salts comprise:
an acid diffusion controlling agent comprising the organic acid anion moiety (i) and the onium cation moiety; and
a radiation-sensitive acid generator comprising an organic acid anion moiety (2) different from the organic acid anion moiety (1) and the onium cation moiety, which generates an acid having a pKa lower than a pKa of an acid generated from the acid diffusion controlling agent by irradiation with radioactive-ray,
wherein at least one of the onium cation moiety in the acid diffusion controlling agent and the onium cation moiety in the radiation-sensitive acid generator is the fluorine-containing onium cation moiety.

3: The radiation-sensitive resin composition according to claim 1, wherein in the formula (1), R¹ comprises an aromatic ring structure.

4: The radiation-sensitive resin composition according to claim 3, wherein the aromatic ring structure comprises an iodine atom.

5: The radiation-sensitive resin composition according to claim 1, wherein in the formula (1), q1 is 1 or 2.

6: The radiation-sensitive resin composition according to claim 1, wherein the fluorine-containing onium cation moiety comprises an aromatic ring structure comprising a fluorine atom.

7: The radiation-sensitive resin composition according to claim 2, wherein a content of the acid diffusion controlling agent is 5 mol % or more and 40 mol % or less with respect to a molar number of the radiation-sensitive acid generator.

8: The radiation-sensitive resin composition according to claim 1, wherein the resin comprises a structural unit comprising a phenolic hydroxyl group.

9: A pattern formation method, comprising:
applying the radiation-sensitive resin composition according to claim 1 directly or indirectly to a substrate to form a resist film;
exposing the resist film to light; and
developing the exposed resist film with a developer.

10: The pattern formation method according to claim 9, wherein the exposing is performed with an extreme ultraviolet ray or an electron beam.
